(19) United States
(12) Patent Application Publication
(10) Pub. No.: US 2003/0148951 A1
(43) Pub. Date: Aug. 7, 2003

(54) Human EMR2, a G-protein coupled receptor from the EGF_TM7 family

(76) Inventors: Lin Hsi-Hsien, Oxford (GB); Siamon Gordon, Oxford (GB); Andrew J. McKnight, Slough (GB); Martin Stacey, Oxford (GB)

Correspondence Address: SCHWEGMAN, LUNDBERG, WOESSNER & KLUTH, P.A., P.O. BOX 2938, MINNEAPOLIS, MN, 55402, US

(21) Appl. No.: 10/270,258

(22) Filed: Oct. 11, 2002

Related U.S. Application Data
(63) Continuation of application No. PCT/GB01/01729, filed on Apr. 17, 2001.

(51) Int. Cl. G01N 33/53; A61K 38/18

(52) U.S. Cl. 514/12; 435/7.1

(57) ABSTRACT

Human EMR2 (EGF-like molecule containing mucin-like hormone receptor-2) a polypeptide of SEG ID NO. 2, and fragments and variants thereof, is a member of the EGF-TM7 family and is useful in the control of wound healing and other conditions associated with neutrophilis and macrophages.

The present invention relates to a protein of the EGF-TM7 family, as well as to nucleotide sequences therefor, preparations thereof and diagnostic methods therefor. Also provided are methods for its preparation and means therefor.

Leukocytes are known to play crucial roles in a range of homeostatic and immune functions. Many of the processes such as growth and maturation, cell rolling and migration, signalling, adhesion, antigen (Ag) uptake, presentation and recognition are mediated by a diverse array of leukocyte cell surface molecules. In general, these cell surface molecules are either single- or multi-span transmembrane proteins of which the extracellular domain is usually composed of one or more definitive structural motifs such as immunoglobulin superfamily (IgSF), epidermal growth factor (EGF), C-type lectin, fibronectin type III, and complement control protein domains. The seven-span transmembrane (7TM) proteins, with more than one thousand members identified to date, represent the largest cell surface protein superfamily in the genome (Bockaert and Pin, 1999; Wess, 1997).

Given the impressive number and signalling capability of the 7TM proteins, it is not surprising that these G protein-coupled receptors (GPCR's) have been found to participate in many aspects of leukocyte biology including chemotaxis, exocytosis and cellular activation. Some of the well-known examples are C5aR (CD88) (Gerard and Gerard, 1994), N-formyl peptide (fMLP) receptor (Boulay et al., 1990; Murphy and McDermott, 1991), interleukin 8 IL-8) receptor (CDw128, CXCR1) (Holmes et al., 1991; Murphy and Tiffany, 1991), and various chemokine receptors including CCR1-CCR5.

Recently, we and others have identified a new subgroup of molecules related to family B GPCR's, designated EGF-TM7, whose members were found to be expressed predominantly on leukocytes (McKnight and Gordon, 1996; McKnight and Gordon, 1998). The EGF-TM7 proteins possess a novel hybrid structure consisting of varying numbers of N-terminal EGF-like domains coupled to a 7TM domain by a mucin-like spacer domain. A number of non-classical family B GPCR-related proteins bearing complex extracellular domains have also been recently described. These include HE6 (Osterhoff et al., 1997), BAI1 (Nishimori et al., 1997), BAI2 and BAI3 (Shiratsuchi et al., 1997), latrophilin (also called CIRL or CLI) (Krasnoperov et al., 1997; Lelianova et al., 1997; Sugita et al., 1998), Celsrl (Hadjantonakis et al., 1998), GPR56/TM7XN 1 (Liu et al., 1999; Zendman et al., 1999), and Ig-Hepta (Abe et al., 1999). To date, four EGF-TM7 molecules have been identified, including human EMR1 (Baud et al., 1995), F4/80 (the probable mouse homologue of human EMR 1, also termed Emr 1) (Lin et al., 1997; McKnight et al., 1996) as well as human and mouse CD97 (Gray et al., 1996; Hamann et al., 1995; Qian et al., 1999).

Investigation of mouse protein F4/80 has given an appreciation of the possible significance of a class of leukocyte-restricted cell surface molecules with unusual structures, the EGF-TM7 proteins. In the review article “The EGF-TM7 family: unusual structures at the leukocyte surface” [McKnight and Gordon, J. Leukocyte Biol. 63, 271-280 (1998)], there is provided an introduction to this class of molecules. The EGF-TM7 proteins comprise a complex structure made up of three subregions; an extracellular NH₂-terminal subregion containing repeated epidermal growth factor (EGF) domains, a membrane-proximal spacer, and seven hydrophobic transmembrane stretches. The hybrid structures suggest possible dual functions as adhesion molecules and as signalling molecules.

Based upon their unique hybrid structures and restricted expression patterns, it has been suggested that the EGF-TM7 molecules may play a role in the immune system by interacting with either cell surface proteins or extracellular matrix proteins, possibly leading to signal transduction via the 7TM domain. Although the possible signalling function of the EGF-TM7 receptors remains to be determined, the recent identification of decay accelerating factor (DAF, CD55) as the cellular ligand of CD97 has proved that they can indeed act as cell adhesion molecules (Hamann et al., 1998; Hamann et al., 1996b).

Chromosome mapping studies have localised human EMR1 and CD97 genes on chromosome 19p13.3 and 19p13.1, respectively Baud et al., 1995; Hamann et al., 1995). Likewise, the mouse F4/80 and Cd97 genes were mapped to the corresponding syntenic regions on mouse chromosomes 17 and 8 (Carver et al., 1999; Lin et al., 1997; McKnight et al., 1997). These results clearly demonstrated that the EGF-TM7 genes have arisen from a common ancestral gene. EMR2, on chromosome 19p13.1, belongs to this family.

Human EGF module-containing mucin-like hormone receptor 1 (EMR1) is the likely human homologue of F4/80. The function of EMR1 remains to be discovered.

EP-A 887,407, published Dec. 30, 1998, relates to a cDNA clone, HAP0167, that encodes a human 7-transmembrane receptor. PCT/WO 00/01818 published on Jan. 13, 2000 relates to a human EMR1-like G protein coupled receptor, EGPCR The respective receptors reported in these patent specifications are from the EGF-TM7 family, and are the same protein. This molecule will also be referred to herein as EMR3. Neither EP-A887,407 nor PCT/WO00/01818 reports the function of EMR3.

The present invention is directed at the provision of an EGF-TM7 protein having an identified function.

In a first aspect, the present invention provides a polypeptide comprising all or part of the sequence of SEQ ID NO. 2, or a variant thereof.

Preferred polypeptides of the present invention possess at least EGF subunit 4 of naturally occurring EMR2, or a variant thereof. More preferably, the polypeptide of the invention comprises 5 EGF subunits, at least one being EGF 4.

For added efficacy, two or more, up to 5, of the EGF subunits, may be tandem or spaced apart repeats of EGF4. Other variations envisaged by the present invention include variants having up to 15 EGF subunits. There are preferably no more than 10 EGF subunits, and preferably no more than 8. Any or all of the subunits may be the equivalent of EGF4.

EGF 4 is that subunit which, when blocked by a specific mAb, prevents binding to EMR2 ligand. Variants of this subunit are possible, but must enable binding to EMR2 ligand, at least in the proper conformation with EGF subunits 1, 2, 3 and 5 of EMR2.

It will be appreciated that the EGF component of EMR2 is important to the extracellular binding properties of EMR^{+ve} cells and, as such, is important to the polypeptides of the present invention where it is desired to take advantage of this property, such as where it is desired to replicate the activity, block it, antagonise it, agonise it, or generate antibodies, for example.

Fragments of the present invention are useful. Such fragments include variants as outlined above which lack one or more of the TM subunits. In particular, where it is desired to provide a soluble polypeptide having the binding properties of EMR2, then it is preferred to eliminate the TM subunits altogether. In such polypeptides, there is generally no need to include the cytoplasmic tail, although this may be retained, if so desired. All or part of the spacer region way be retained, and may serve to provide some stability to the EGF region.

It may also be desirable to provide other sequences attached to the polypeptides of the present invention, such as to alter solubilisation characteristics, or to provide any other desired properties. If these block the activity of the EMR2 region, then it is preferred that they be removable, such as by being cleaved in vivo.

It will be appreciated that the present invention extends to polynucleotides, and variants thereof, encoding any polypeptide of the present invention.

The present invention also extends to vectors, especially expression vectors, comprising such polynucleotides, to cells containing such vectors, to systems for generating polypeptides of the invention from such cells, and to the products of such systems.

Antibodies specific for any part of EMR2 are part of the present invention, and may be raised and used as described below.

Agonists for EMR2 may be selected by means well known in the art, and form a part of the invention. Likewise, antagonists for EMR2 may be selected by means well known in the art, and also form a part of the invention.

It may be desired to block naturally occurring EMR2, and this can be achieved, for example, by introducing anti-EMR2 mAb to the system, or by saturating the system with soluble EMR2 fragment, for example. Where the effect of EMR2 is desired, without the cells expressing it, then introducing a suitable polypeptide of the invention to the site is useful.

The present invention is concerned with a protein which we have designated EMR2 (SEQ ID NO. 2). The sequence for EMR2, encoding an EGF-TM7 protein, is to be found in the GenBank Data Bank under the accession number AF1 14491. Sequence analysis reveals that EMR2 has an extracellular NH₂-terminal subregion containing five EGF domains, a membrane-proximal spacer, seven hydrophobic transmembrane (TM) stretches, and a cytoplasmic tail, along with multiple sites for N- and O-glycosylation. The potential exists for alternative splicing of mRNA transcripts, giving rise to, for example, multiple isoforms with different combinations and numbers of the EGF domains.

We have found that EMR2 plays an important role in cell adhesion. It is expressed most abundantly in peripheral blood leukocytes, followed by high levels of expression in spleen and lymph nodes, and appears to be restricted to cells of certain hematopoietic lineages. In particular, EMR2 is very strongly expressed in polymorphonuclear cells and at lower levels in freshly isolated strongly-adhering monocytes, but probably not in weakly adherent monocytes or peripheral blood lymphocytes. Furthermore, EMR2 expression in monocytes is upregulated following maturation.

We have further found that the extracellular EGF domains of EMR2 will bind to monocyte-derived macrophages, indicating a crucial role in cell-cell adhesion.

In accordance with this invention, we provide a polypeptide which is a purified precursor EMR2 protein comprising the amino acid sequence of FIG. 1 (SEQ ID NO. 2) or a polypeptide fragment thereof. The polypeptides of this invention thus include the naturally occurring protein, as well as biologically active fragments. Such fragments include a mature EMR2 which might be formed by cleavage at residue Thr²³ (SEQ ID NO. 20). The EMR2 polypeptide sequence is encoded by the sequences of SEQ ID NO'S 1 and 19.

The EGF-TM7 proteins (EMR1, F4/80 and CD97) constitute a recently defined class B GPCR subfamily that are predominantly expressed on leukocytes. These molecules possess N-terminal EGF-like domains coupled to a seven-span transmembrane (7TM) moiety via a mucin-like spacer domain. Genomic mapping analysis has suggested a possible EGF-TM7 gene family on human chromosome 19p13 region. The present invention relates to a new member of the EGF-TM7 family, EMR2, which shares strikingly similar molecular characteristics with CD97. The nucleotide sequence for EMR2 has been available on the GenBank™ Data Bank under the accession number AF114491 since Jan. 1, 2000.

EMR2 is closely related to human CD97 (FIGS. 2, 3). EMR2 and CD97 share almost identical EGF-like domains, express similar alternatively-spliced transcripts, map close to each other on chromosome 19p13.1 and are the probable products of a recent gene duplication event.

Although both sequences are strikingly similar, EMR2 and CD97 display significant differences. EMR2 and CD97 are both expressed predominantly in immune tissues (PBL, spleen, lymph node, bone marrow and foetal liver) with expression contributed mainly by granulocytes and monocytes/Mφs. CD97, however, is also expressed in other tissues and cell types (FIG. 4). The broader expression pattern of CD97 suggests that it has a more general function than EMR2, whose function might be restricted to granulocytic and monocyte/Mφ lineages.

In addition to mapping closely to CD97 on human chromosome 19p13.1, EMR2 also contains a total of five tandem EGF-like domains and expresses similar protein isoforms consisting of various numbers of EGF-like domains as a result of alternative RNA splicing. Furthermore, EMR2 and CD97 exhibit highly homologous EGF-like domains and share identical gene organisation, suggesting that both genes are the products of a recent gene duplication event. The homologous EGF-like domains enable the identification of both EMR2 and CD97 by mAbs raised against the first EGF-like domain of CD97, whereas mAbs directed against the extracellular spacer domain of CD97 are able to differentiate the two proteins.

Both EMR2 and CD97 are highly expressed in immune tissues. However, unlike CD97, which is ubiquitously expressed in most cell types, EMR2 expression is restricted to monocytes/Mφ and granulocytes. EMR2 fails to interact with CD55, the cellular ligand for CD97, but appears to bind an EMR2 ligand. EMR2 may, therefore, have a unique function in cells of monocyte/Mφ and granulocytes lineages. In addition, it weakly interacts with CD55, which may provide a fine level of control in interactions with CD55^{+ve} cells.

We have demonstrated that the CD97-CD55 interaction is not glycosylation dependent, i.e., is glycosylation independent. Accordingly, it is not necessary to glycosylate polypeptides of the present invention in order for them to possess biological activity. This is a surprising advantage. Nevertheless, it will be appreciated that the present invention envisages glycosylated EMR2 and its related peptide derivatives, as defied and described herein.

Although cell-based binding assays show no detectable CD55-EMR2 interaction, SPR assays detect a weak but specific interaction between CD55 and EMR2. It is possible that the much weaker CD55-EMR2 interaction has fallen beyond the detection limit of the cell-based assay systems, which are less sensitive than SPR. Without being bound by theory, it seems probable that, given high enough levels of cell-surface CD55 proteins, one would be able to detect the CD55-EMR2 interaction using cell-based assay systems. Since both CD97 and EMR2 are predominantly expressed by granulocytes, monocytes and macrophages, this provides a mechanism whereby the CD55-binding ability of these important immune cells could be regulated by the cell surface expression levels of a pair of closely related EGF-TM7 proteins, EMR2 providing a more subtle interaction. Accordingly, modulation of the EMR2 interaction in accordance with the present invention could be used to modify the activity of these cells, where desired.

The Ca²⁺-dependence of the CD97-CD55 interaction indicates that the Ca²⁺ binding sites within the EGF-2 and 5 domains of CD97 are crucial for intermolecular interactions. Structural data from a fibrillin-1 cbEGF pair have shown that Ca²⁺-binding is required for the maintenance of interdomain rigidity. As a consequence tandem repeats of cbEGF domains or EGF-cbEGF domains with similar conservation of residues are predicted to form extended rod-like strunctures which present specific protein surfaces for protein-protein interactions. Inspection of the sequences of EGF-cbEGF and cbEGF-cbEGF pairs from CD97 and EMR2 suggest they are of the fibrillin-1 or class I type, since they have one residue between the last Cys residue of the N-terminal cbEGF and the first calcium binding residue of the C-terminal cbEGF. In addition, hydrophobic packing residues also implicated in maintaining the rod-like conformation of fibrillin-1 cbEGFs are conserved in CD97 and EMR2. Calcium binding to cbEGF domains is, therefore, probably critical in maintaining CD97-CD55 interaction by sustaining an overall rod-like structure of the 3 EGF domains. Accordingly, it is preferable to provide preparations of peptides of the present invention which comprise sufficient calcium to have such a rigidifying effect, especially where it is desired that the peptide have a physiological effect. However, it will be appreciated that calcium will generally be acquired by the peptide in vivo, if none is supplied in the preparation.

The complete abrogation of the CD55/97 interaction in the absence of Ca²⁺ suggests that the protein surface on CD97 recognised by CD55 extends over the domain 1-2 boundary rather than being localised on a single domain. This is confirmed by the locations of the amino acid changes; within domains 1 and 2, which differentiate CD97 and EMR2. To better understand how any one of these amino acid changes causes a reduction in CD55 binding, effects which occur directly, due to alteration of a side chain which previous contacted CD55 or insertion of a bulky group within the interface have to be distinguished from those changes which disturb binding indirectly by producing a more long-range alteration in structure. A small number of missense mutations in cbEGF domains associated with human disease are localised in variable loop regions and it has been predicted that in fibrillin-1 these mutations occur at sites used for protein-protein interactions.

EGF modules are found to be highly consistent in structure, with the positions of alpha carbons varying by less than 2.5 Å, when all EGF structures determined to date are compared. The known structure of an EGF pair can, therefore, be used to predict the locations of the EMR2 mutations on the first two domains of CD97. Mapping of the three amino acid changes between CD97 and EMR2 onto the three dimensional structure of fibrillin-1 domains 32 and 33 indicates that they are likely to be located in these variable loops and may, therefore, directly participate in the interface with CD55. It remains possible, however, that the mode of action for the Leu to Pro substitution at position 71 is indirect, as a Pro at this position is implicated in stabilising the hydrophobic core of a cbEGF domain from factor IX.

Recognition of both EMR2 and CD97 proteins by mAbs BL-Ac/F2 and CLB-CD97/1, directed against EGF-like domain 1 of CD97, was expected but raises doubts concerning the true expression pattern of the CD97 antigen. Since most reports regarding the tissue expression patterns of the CD97 antigen have mainly used BL-Ac/F2 and CLB-CD97/1 mAbs (Eichler et al., 1994; Eichler et al., 1997; Hoang-Vu et al., 1999), the distinction between EMR2^{+ve} and CD97^{ve} cells within tissues and complex populations of cells would, unwittingly, have been confused, as the existence of EMR2 was not suspected, at that time.

Although it is logical to assume that these two molecules serve as backup for each other, the inability of EMR2 to interact with CD55 has shown that this is untrue. It is also interesting to note that the CD97-CD55 interaction is very specific, as demonstrated by the three amino acid differences in the EGF-like domains of the shortest EMR2 and CD97 isoforms.

The similar stuctrual feature of the EMR2 protein to those of CD97 suggest that EMR2 is very likely to function as a cellular adhesion molecule as well. Possible ligand candidates include the regulators of complement activation, such as CD46 (membrane cofactor protein, MCP), CD35 (CR1), CD21 (CR2) and C4 binding protein (C4bp), all of which contain short consensus repeats similar to those found in CD55 (Liszewski et al., 1996).

Western blot analysis of Mφ cell lysate treated with or without various glycosidases has revealed that EMR2 is a heavily glycosylated cell surface protein. In FIG. 11, we show that 2A1 mAb specifically recognised EMR2 [in FIG. 11-A) Western Blotting. Cell lysate from mock transfected HEK-293T cells (lane 1), cells transfected with EMR2 (12), EMR2 (125), EMR2 (1235), EMR2 (12345), or CD97 (12345) expression constructs (lanes 2-6 respectively) were separated on SDS-PAGE, Western blotted and probed with 2A1 mAb. Only EMR2 protein isoforms, with different molecular masses, were recognised by 2A1 mAb. B, C, D) Immunocytochemistry. CHO-K1 cells were transiently transfected with CD97 (12345) (B), EMR2 (12345) (C), or without expression construct (D), fixed and stained with 2A1. Again, only EMR2 -transfected cells showed a positive reactivity].

In addition, immunohistochemistry has shown that EMR2 is expressed in certain tissue Mφ and myeloid cells including neutrophil and DC in normal and diseased tissues such as psoriatic skin and tonsils.

By adapting a well-established technique (Brown, M. H., et al., (1995) Eur J Immunol 25(12), 3222-8; Brown, M. H., et al., (1998) J Exp Med 188(11), 2083-90), we have successfully identified a putative cellular ligand for EMR2 using multivalent forms of the extracellular domain of the EMR2 protein as probes. The EMR2-EMR2 ligand interaction is Ca²⁺-dependent and mediated in an isoform-specific manner. Only the EMR2 (EGF-1,2,3,4,5) isoform, but not the other isoforms, binds the putative ligand (FIG. 12).

FIG. 12 shows FACS analysis. A) Schematic representation of biotinylated soluble EMR2 proteins and the protein-fluorescent bead complex. B) Western blot demonstrates the production of biotinylated EMR2 protein isoforms, which was probed with ExtrAvidin-HRP to detect the extent of biotinylation. C) FACS analysis showing the expression of the putative EMR2-ligand on day 3-human monocyte-derived Mfs. Note the binding of the protein-bead probes to cells is isoform-specific (thin line, beads alone control; thick line, EMR2 protein isoform). Addition of 5 mM EGTA to the reaction abolished the binding of EMR2(12345)-beads to cells (dash line), indicating that the binding is Ca²⁺-dependent.

Addition of mAbs specific to the 4^{th} EGF-like domain of EMR2 completely abolished the EMR2-EMR2 ligand interaction indicating that the EGF-like domain 4 is either an essential part of the ligand-binding domain or is the ligand-binding domain. The putative EMR2-ligand is found to be expressed on human monocyte-derived Mφ as well as human embryonic kidney fibroblast (HEK-293T), HeLa and hepatoma Hep3B, but not on other primary blood cells (erythrocytes, resting and activated T and B cells and granulocytes) and haematopoietic cell lines (HL60, THP-1, etc). In addition to the human cells, some rodent fibroblast cell lines (NIH3T3, CHO-K1) also show strong EMR2-ligand expression, suggesting that the EMR2-EMR2 ligand interaction may have physiological functions of evolutionary significance (Table 1).

TABLE 1
EMR2-Ligand Expression Analysed by
Biotinylated EMR2-Fluoresceut Bead Complex

| | (EMR2-Ligand) |
|---|---|
| | Binding Affinity |
| Primary Cells | |
| PBLymphocytes (B + T)^{a} | − |
| Monocytes | +/− |
| Monocyte-derived Mφs (Day | ++ |
| 1-5) | |
| Neutrophils | − |
| Erythtocytes | − |
| Cell Lines | |
| HL-60^{b} | − |
| THP-1^{b} | − |
| MonoMac6^{b} | − |
| U937^{b} | − |
| K562^{b} | − |
| HEL^{b} | − |
| Daudi | − |
| H9 | − |
| Jurkat | − |
| A20 | − |
| HEK293T | ++++ |
| Hep3B | + |
| HepG2 | +/− |
| HeLa | +++ |
| NIH3T3 | ++++ |
| RAW | + |
| COS-7 | ++++ |
| CHO-K1 | ++++ |
| SVAREC | ++++ |

^{a}Cells were treated with or without PHA-L (2 μg/ml for 2, 6 and 24 hrs)
^{b}Cells were treated with or without PMA (10 ng/ml for 2 days)

To gain more information on the in situ tissue distribution of the putative EMR2 ligand, frozen mouse tissue sections were overlaid with the fluorescent multivalent probes, washed and fixed for examination. The binding pattern revealed a “connective tissue”-type appearance, including vascular structures in most tissues such as heart, liver and spleen, lamina propria and submucosa area of small gut and stomach, interstitial connective tissue surrounding the seminiferous tubules of the testis, etc. (FIGS. 13, 14 and Table 2).

FIG. 13 shows tissue staining by the EMR2 protein-fluorescent bead complex. Only EMR2(12345)-fluorescent beads (B, D) but not EMR2(125)-fluorescent beads (A, C) or fluorescent beads alone (not shown) bind to mouse ear skin (A, B) and oesophagus (C, D) sections. The images in A and C were over-exposed to show the outline of the tissues. No binding of beads was found.

FIG. 14 shows tissue staining patterns of the EMR2-ligand. Frozen mouse tissue sections of small intestine (A), oesophagus (B), testis (C) and ovary (D) were stained with EMR2(12345)-fluorescent beads, washed and photographed to show the distribution pattern of the EMR2-ligand in these tissues. Lamina propria and submucosa of small intestine (A), lamina propria and adventitia of oesophagus (B), interstitial connective tissues immediately surrounding the seminiferous tubules (C) and cortex stroma and medulla of ovary (D) show the strongest binding.

TABLE 2
Tissue Distribution of the EMR2-ligand Detected by
the Biotinylated EMR2-Fluorescent Bead Binding Assay

| Tissues | Reactivity |
|---|---|
| Brain | − |
| Heart | + |
| Kidney | + |
| Skin | ++ |
| Liver | +/− |
| Spleen | + |
| Thymus | +/− |
| Lymph node | − |
| Lung | − |
| Small Intestine | ++ |
| Smooth muscle | +++ |
| Tendon | +++ |
| Uterus | +++ |
| Ovary | +++ |
| Testis | +++ |
| Pancreas | ++ |
| Oesophagus | ++++ |

As EMR2 is predominantly expressed in professional phagocytes (monocytes/macrophages and neutrophils), the interaction between EMR2 and EMR2-ligand thus may dictate how neutrophils and monocytes/Mφs migrate through tissues and modulate immune and inflammatory responses by activating or deactivating the cells.

In particular, neutrophils are implicated in acute inflammation such as might occur with an injury or with an infection such as meningitis and pneumonia; chronic inflammation such as rheumatoid arthritis, chronic tissue damages; septic shock; repair and auto-immune disease processes; atherosclerosis; diabetes; Alzheimer's disease; as well as in more general processes such as killing of targets by degranulation; chemotaxis and leukocyte recruitment; induction and effector mechanism of innate and acquired immunity. Macrophages are also implicated in clotting; fibrinolysis; intravascular coagulation; thrombosis and embolism; wound repair and angiogenesis. Macrophage-neutrophil interactions may also be involved in the development of granulocytes, thus a possible role in haematopoiesis and blood disorders such as aneutropenia and agranulocytosis as well as myeloid leukaemia. Interaction between mature macrophages and haematopoietic precursors may be important in anaemia.

Specific areas of potential implications include the migration, retention and activation/de-activation of phagocytes, which may lead to acute or chronic inflammation, tissue damage and auto-immune diseases.

The interaction between EMR2 on phagocytes and the EMR2 ligand on connective tissues may be involved in general disorders of connective tissue such as wound healing, vascular malfunction or congenital diseases such as hereditary hemorrhagic telangiectasia (HHT) and Marfan syndrome. Tumour formation, metastis, surveillance and control may also be influenced by the EMR2-EMR2 ligand interaction. Further, since both EMR2 and EMR2 ligand are expressed in Mφ, the formation of Mφ giant cells in bacterially-induced granuloma could potentially also result from the interaction between EMR2 and EMR2 ligand.

It is, therefore, envisioned that possible therapeutic intervention for such types of pathological disorders could be achieved by modulating EMR2-EMR2 ligand interaction. Examples of specific targets include stimulation of wound healing and post-infections tissue repair, inhibition of excess tissue repair, such as fibrosis in lung and liver, inhibition of tissue injury such as arthritis in joints. The possible therapeutic agents include soluble EMR2, soluble EMR2 ligand or small peptides that affect the EMR2-EMR2 ligand interaction.

There is the probability that the intracellular cytoplasmic tail subregion of EMR2 has a signalling function, and this knowledge may form the basis of further treatments in accordance with this invention.

Unexpectedly, a splice variant resulting from the bypass of exon 12 has been identified and predicted to encode a soluble EMR2 molecule due to a frameshift, which introduces a stop codon immediately before the first TM domain (FIG. 5C). Such a soluble molecule is a preferred feature of the present invention. It will be appreciated that it is convenient to use the naturally occurring variant, but other soluble forms of EMR2 will also be readily apparent to those skilled in the art, and can be prepared as desired.

Soluble EMR2 and its encoding nucleotide sequence may, for example, be a mutant, variant, allele or isoform of any form of EMR2 lacking the TM regions, and may comprise less than 5 EGF regions, for example, although it is preferred that at least EGF4 is present. In addition, it is generally preferred that the majority of the linker be present.

Soluble EMR2 is useful as a blocker for naturally occurring EMR2, and can be used to control fibrosis, for example. In addition, soluble EMR2 may be used in obtaining antibodies, preferably in non-human animals, or may be used as a passive or active mammalian vaccine, especially in humans.

In addition, a further splicing event resulting from the utilization of different splicing acceptor sites in exon 12 has also been identified. As a result, an EMR2 transmembrane molecule with an 11 amino acid deletion in the spacer domain is predicted (data not shown). Such a deletion mutant is another preferred feature of the invention. Similar alternative splicing of exons encoding the spacer and transmembrane regions of CD97 has also been observed. To date we have identified at least 7 alternative EMR2 transcripts by RT-PCR in peripheral blood leukocytes (FIG. 5B).

The invention extends to any peptide or polypeptide, which terms are used interchangeably herein, except where otherwise required, which has substantial identity with precursor or mature EMR2 or a fragment thereof. Polypeptides with substantial identity include variants, such as allelic variants.

A “variant” as the term is used herein may refer to a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

Within the variants we include altered polypeptides and polynucleotides. “Altered” nucleic acid sequences encoding EMR2 include those sequences with deletions, insertions, or substitutions of different nucleotides, resulting in a polynucleotide the same as EMR2 or a polypeptide with at least one fractional characteristic of EMR2. Included with this definition are polymorphisms which may or may not be readily detectable using a particular oligonucleotide probe of EMR2, and improper or unexpected hybridisation to allelic variants, with a locus other than the normal chromosomal locus for the polynucleotide sequence encoding EMR2. The encoded protein may also be “altered,” and may contain deletions, insertions, or substitutions of amino acid residues which produce a silent change and result in a fractionally equivalent EMR2. Deliberate amino acid substitutions may be made on the basis of similarity in polarity, charge, solubility, hydrophobicity, hydrophilicity, and/or the amphipathic nature of the residues, as long as the biological or immunological activity of EMR2 is retained. For example, negatively charged ammo acids may include aspartic acid and glutamic acid, positively charged amino acids may include lysine and arginine, and amino acids with uncharged polar head groups having similar hydrophilicity values may include leucine, isoleucine, and valine; glycine and alanine; asparagine and glutamine; serine and threonine; and phenylalanine and tyrosine.

Fragments of EMR2 may take various forms, and are also included in the invention. A fragment is a polypeptide having an amino acid sequence that entirely is the same as part, but not all, of the amino acid sequence of EMR2, or a variant thereof. Fragments maybe “free-standing”, or comprised within a larger polypeptide of which they form a part or region, most preferably as a single continuous region. Representative examples of polypeptide fragments of the invention include, for example, fragments from about amino acid number 1-20, 21-40, 41-60, 61-80, 81-100, and 101 to the end of EMR2 polypeptide. In this context “about” includes the particularly recited ranges larger or smaller by several, 5, 4, 3, 2 or 1 amino acid at either extreme or at both extremes.

Preferred fragments include, for example, truncation polypeptides having the amino acid sequence of the EMR2 polypeptide, except for deletion of a continuous series of residues that includes the amino terminus, or a continuous series of residues that includes the carboxyl terminus or deletion of two continuous series of residues, one including the amino terminus and one including the carboxyl terminus. Also preferred are fragments characterised by structural or functional attributes such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding regions, and high antigenic index regions. Other preferred fragments are biologically active fragments. Biologically active fragments are those that mediate receptor activity, including those with a similar activity or an improved activity, or with a decreased undesirable activity. Also included are those that are antigenic or immunogenic in an animal, especially in a human if it is desired to block EMR2 binding, for example.

Preferably, all of these polypeptide fragments retain the biological activity of the receptor, including antigenic activity. Among the most preferred fragments are those containing EGF4. Variants of the defined sequence and fragments also form part of the present invention. Preferred variants are those that vary from the referents by conservative amino acid substitutions—i.e., those that substitute a residue with another of like characteristics. Typical such substitutions are among Ala, Vat, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, or 1-2 amino acids are substituted, deleted, or added in any combination.

Substantially identical polypeptides form a preferred embodiment of the present invention, and include those with variation in at least one part of the molecule, with the option of retaining unchanged another part of the molecule. Thus, there can be differences in one or more of the EGF subregions, in the spacer subregion, in the transmembrane subregion, or in the cytoplasmic tail. The degree of identity may vary depending on the function which the polypeptide is intended to mimic. Thus, for example, if the substantially identical polypeptide is to mimic the cell-cell binding capability of EMR2, then it may be preferable to retain unchanged the EGF subregions, while adopting changes in one or more of the other subregions, if desired. The changes may include deletion of such subregions, thereby giving active fragments of this invention.

The polypeptides with substantial identity preferably have at least 80% identity with a corresponding polypeptide which is precursor or mature EMR2 or a fragment thereof. More preferably there is at least 90% identity, such as at least 95% or at least 98% identity. Sequence identity is suitably determined by a computer programme, though other methods are available. We prefer that identity is assessed using the Gap program from Genetic Computer Group (1994), (see Program Manual for the Wisconsin Package, Version 8, Madison Wis., USA).

“Identity” is a measure of the identity of nucleotide sequences or amino acid sequences. In general the sequences are aligned so that the highest order match is obtained. “Identity” per se has an art-recognised meaning and can be calculated using published techniques. See: e.g.: (COMPUTATIONAL MOLECULAR BIOLOGY, Lesk, A. M., ed., Oxford University Press, New York, 1988; BIOCOMPUTING: INFORMATICS AND GENOME PROJECTS, Smith, D. W., ed.: Academic Press, Now York, 1993; COMPUTER ANALYSIS OF SEQUENCE DATA, PART 1, Griffin, A. M., and Griffin, H. G., eds., Humana Press, New Jersey, 1994; SEQUENCE ANALYSIS IN MOLECULAR BIOLOGY, von Heinje, G., Academic Press, 1987; and SEQUENCE ANALYSIS PRIMER, Gribskov, M. and Devereux, J., eds., M Stockton Press, New York, 1991).

While there exist a number of methods to measure identity between two polynucleotide or polypeptide sequences, the term “identity” is well known to skilled artisans (Carillo, H., and Lipton, D., SIAM J Applied Math (1988) 48:1073). Methods commonly employed to determine identity or similarity between two sequences include, but are not limited to, those disclosed in Guide to Huge Computers, Marlin J. Bishop, ed., Academic Press, San Diego, 1994, and Carillo, H., and Lipton, D., SIAM J Applied Math (1988) 48:1073. Methods to determine identity and similarity are codified in computer programs. Preferred computer program methods to determine identity and similarity between two sequences include, but are not limited to, GCS program package (Devereux, J., et al., Nucleic Acids Research (1984) 12(1):387); BLASTP, BLASTN, FASTA (Atschul, S. F. et al., J Mol. Biol. (1990) 215:403).

As an illustration, by a polynucleotide having a nucleotide sequence having at least, for example, 95% “identity” to a reference nucleotide sequence of SEQ ID NO: 1 is intended that the nucleotide sequence of the polynucleotide is identical to the reference sequence except that the polynucleotide sequence may include up to five point mutations per each 100 nucleotides of the reference nucleotide sequence of SEQ ID NO: 1. In other words, to obtain a polynucleotide having a nucleotide sequence at least 95% identical to a reference nucleotide sequence, up to 5% of the nucleotides in the reference sequence maybe deleted or substituted with another nucleotide, or a number of nucleotides up to 5% of the total nucleotides in the reference sequence may be inserted into the reference sequence. These mutations of the reference sequence may occur at the 5 or 3 terminal positions of the reference nucleotide sequence or anywhere between those terminal positions, interspersed either individually among nucleotides in the reference sequence or in one or more contiguous groups within the reference sequence.

Similarly, by a polypeptide having an amino acid sequence having at least, for example, 95% “identity” to a reference amino acid sequence of SEQ ID NO: 2 is intended that the amino acid sequence of the polypeptide is identical to the reference sequence except that the polypeptide sequence may include up to five amino acid alterations per each 100 amino acids of the reference amino acid of SEQ ID NO: 2. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

The polypeptides of this invention may be embedded within a longer sequence, in the form of a fusion protein, for example. Thus, the polypeptides may be in the form of the “mature” protein, or may be part of a larger protein, such as a fusion protein. It is often advantageous to include an additional amino acid sequence which contains secretory or leader sequences, pro-sequences, sequences which aid in purification, such as multiple histidine residues, or an additional sequence for stability during recombinant production.

The polypeptides of this invention which are fragments preferably retain the cell binding, signalling or other desired function of EMR2. Typical fragments of this invention include isoforms with fewer than five of the extracellular EGF domains; soluble molecules comprising part or all of the membrane-proximal spacer, and one or more of the extracellular EGF domains; and an EMR2 molecule with an amino acid deletion in the spacer subregion, with or without deletions in the TM or EGF regions. Specific examples of such fragments are mentioned later in the text. The fragments suitably comprise at least 10 amino acids, more preferably at least 20 amino acids, and typically at least 40 amino acids.

In one important aspect for investigating EMR2 activity, the present invention provides beads or other forms of substrate carrying a fragment of this invention, suitably comprising one or more functional sequences, preferably with a linker sequence between the bead and the functional sequence. The linker sequence may comprise all or a part of the spacer subregion of a polypeptide of this invention. The fragment can be linked to the substrate using conventional procedures for so securing an amino acid sequence, including biotin-avidin coupling. In one variation, the substrate carries a naturally occurring sequence, for example an EGF sequence, such as the EGF sequences which occur in the different isoforms of EMR2. In an alternative variation, multiple polypeptide sequences are secured to the substrate as an array.

The polypeptides of this invention may be fully or partially glycosylated, or may be unglycosylated.

The polypeptides are preferably in purified form. In one aspect, they have a naturally occurring, sequence and are isolated polypeptides.

The invention further involves the following aspects, including a polynucleotide, RNA or DNA, encoding a polypeptide of this invention, including fragments;

a polynucleotide which hybridises to such a polynucleotide or is complementary to such a polynucleotide;

a polynucleotide comprising at least a fragment of the nucleic acid sequence of FIG. 1 (SEQ ID NO. 1 and SEQ ID NO. 19);

variants of these polynucleotides including polynucleotides with substantial identity and including altered polynucleotides;

a cell which is EMR2⁺ or which carries another transmembrane polypeptide of this invention;

an expression vector including a polynucleotide of this invention and which permits expression of a polypeptide of this invention;

a host cell containing such a vector;

a process for preparing a polypeptide of this invention, which comprises culturing the host cell and recovering the protein or polypeptide fragment;

an antibody or antibody fragment which binds to a polypeptide of this invention;

an agonist or an antagonist of a polypeptide of this invention;

a pharmaceutical composition comprising a polypeptide, a polynucleotide, an antibody, an agonist or an antagonist of this invention, together with a pharmaceutically acceptable carrier or diluent;

a diagnostic method involving assessing expression of EMR2 protein or involving identification of a mutation in DNA encoding such protein;

a diagnostic method involving use of a polynucleotide of this invention as a probe for cells or other samples which might express EMR2 protein;

a method of treatment involving administering as appropriate a polypeptide, a polynucleotide, an antibody, an agonist or an antagonist of this invention; and

the use of a polypeptide, a polynucleotide, an antibody, an agonist or an antagonist of this invention in the preparation of a medicament for use in a method of therapy.

The polynucleotides may encode for the polypeptide of this invention, and in particular the invention provides a polynucleotide comprising at least a fragment of the nucleic acid sequence of FIG. 1 (SEQ ID NO: 1). The polynucleotides of this invention can be provided without additional sequences, or may further include other sequences in reading frame alignment, such as one or more of a leader, secretory, marker or other sequence. Untranslated regions are present in the DNA sequence of FIG. 1 (SEQ ID NO: 1), and these or other untranslated regions, which may be selected for other functions, may be included or omitted from a polynucleotide of this invention. The polynucleotides are preferably in purified form. In one aspect, they have a naturally occurring sequence and are isolated polynucleotides.

Variant polynucleotides include those arising from degeneracy in the genetic code.

Recombinant polypeptides produced from engineered polynucleotides are also part of this invention.

As with the polypeptides of this invention, the polynucleotides of this invention can also be used to investigate the role of EMR2.

A hybridising polynucleotide preferably hybridises under stringent conditions to such a polynucleotide, the stringent conditions being selected to reflect a degree of substantial identity as discussed in preceding paragraphs. For example, the conditions preferably give hybridisation when there is about 80% identity.

More specifically, stringency, as it is commonly used in the art, is defined in Wahl, G. M. and S. L. Berger (1987; Methods Enzymol. 152:399-407) and Kimmel, A. R. (1987; Methods Enzymol. 152:507-511). For example, stringent salt concentration will ordinarily be less than about 750 mM NaCl and 75 mM trisodium citrate, preferably less an about 500 mM NaCl and 50 mM trisodium citrate, and most preferably less than about 250 mM NaCl and 25 mM trisodium citrate. Low stringency hybridisation can be obtained in the absence of organic solvent, e.g., formamide, while high stringency hybridisation can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridisation time, the concentration of detergent, e.g., sodium dodecyl sulphate (SDS), and the inclusion or exclusion of carrier DNA, are well known to those skilled in the art. Various levels of stringency are accomplished by combining these various conditions as needed. In a preferred embodiment, hybridisation will occur at 30° C. in 750 mM NaCl, 75 mM trisodium citrate, and 1% SDS. In a more preferred embodiment, hybridisation will occur at 37° C. in 500 mM NaCl, 50 mM trisodium citrate, 1% SDS, 35% formamide, and 100 μg/ml denatured salmon sperm DNA (ssDNA). In a most preferred embodiment, hybridisation will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The washing steps which follow hybridisation can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl 15 mM trisodium citrate, and 0.1% SDS. In a most preferred embodiment wash steps will occur at 68° C. in 15 mM NaCl, 1.5 mM trisodium citrate, and 0.1% SDS. Additional variations on these conditions will be readily apparent to those skilled in the art.

The expression vectors of this invention include a polynucleotide of this invention and/or encode for a polypeptide of this invention, and permit expression of a polypeptide of this invention. The construction of such vectors is well documented in the literature.

Thus, the present invention also relates to vectors which comprise a polynucleotide or polynucleotides of the present invention, and host cells which are genetically engineered with vectors of the invention and to the production of polypeptides of the invention by recombinant techniques. Cell-free translation systems can also be employed to produce such proteins using RNA's derived from the DNA constructs of the present invention.

For recombinant production, host cells can be genetically engineered to incorporate expression systems or portions thereof for polynucleotides of the present invention. Introduction of polynucleotides into host cells can be elected by methods described in many standard laboratory manuals, such as Davis et al., BASIC METHODS IN MOLECULAR BIOLOGY (1966) and Sambrook et al., MOLECULAR CLONING: A LABORATORY MANUAL, 2nd Ed., Cold Spring Harbor Laboratory Press, Cold Spring Harbor, N.Y. (1989), such as calcium phosphate transfection, DEAE-dextran mediated transfection, transfection, microinjection, cationic lipid-mediated transfection, electroporation, transduction, scrape loading, ballistic introduction or infection.

Representative examples of appropriate hosts include bacterial cells, such as streptococci, staphylococci, E. coli, Streptomyces and Bacillus subtilis cells; fungal cells, such as yeast cells and Aspergillus cells; insect cells such as Drosophila S2 and Spodoptera S19 cells; animal cells such as CHO, COS, HeLa, C127, 3T3, BHK, HEK 293 and Bowes melanoma cells; and plant cells.

A great variety of expression systems can be used. Such systems include, among others, chromosomal, episomal and virus-derived systems, e.g., vectors derived from bacterial plasmids, from bacteriophage: from transposons, from yeast episomes, from insetion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage geetic elements, such as cosmids and phagemids. The expression systems may contain control regions that regulate as well as engender expression. Generally any system or vector suitable to maintain, propagate or express polynucleotides to produce a polypeptide in a host maybe used. The appropriate nucleotide sequence may be inserted into an expression system by any of a variety of well-known and routine techniques, such as, for example, those set forth in Sambrook et al., MOLECULAR CLONING, A LABORATORY MANUAL (supra).

For secretion of the translated protein into the lumen of the endoplasmic reticulum, into the periplasmic space or into the extracellular environment appropriate secretion signals may be incorporated into the desired polypeptide. These signals may be endogenous to the polypeptide or they may be heterologous signals.

If the polypeptide is to be expressed for use in screening assays, generally, it is preferred that the polypeptide be produced at the surface of the cell. In this event, the cells may be harvested prior to use in the screening assay. If the polypeptide is secreted into the medium, the medium can be recovered in order to recover and purify the polypeptide; if produced intracellularly, the cells must first be lysed before the polypeptide is recovered.

Polypeptides can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxyapatite chromatography and lectin chromatography. Most preferably, high performance liquid chromatography is employed for purification. Well known techniques for refolding protein may be employed to regenerate active conformation when the polypeptide is denatured during isolation and or purification.

The antibodies of this invention bind to a polypeptide of this invention, and can be made by available techniques. The antibodies can be chimeric or humanised, and may take the form of a fragment, notably a binding domain of the antibody. They may have antagonistic activity, or bind in a manner which does not antagonise the functional activity of the polypeptide.

An antagonist of the EMR2 polypeptide may be produced using methods which are generally known in the art. In particular, purified EMR2 polypeptide (also simply referred to as EMR2 herein) may be used to produce antibodies or to screen libraries of pharmaceutical agents to identify those which specifically bind EMR2. Antibodies to EMR2 may also be generated using methods that are well known in the art. Such antibodies may include, but are not limited to, polyclonal, monoclonal, chimeric, and single chain antibodies, Fab fragments, and fragments produced by a Fab expression library. Neutralising antibodies (i.e., those which inhibit dimer formation) are especially preferred for therapeutic use.

For the production of polyclonal antibodies, various hosts including goats, rabbits, rats, mice, humans, and others may be immunised by injection with EMR2 or with any fragment or oligopeptide thereof which has immunogenic properties. Rats and mice are preferred hosts for downstream applications involving monoclonal antibody production. Depending on the host species, various adjuvants may be used to increase immunological response. Such adjuvants include, but are not limited to, Freund's, mineral gels, such as aluminium hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable. The methods for antibody production and analysis are described in Harlow, E. and Lane, D. (1988; Antibodies: A Laboratory Manual, Cold Spring Harbor Laboratory, Cold Spring Harbor N.Y.).

It is preferred that the oligopeptides, peptides, or fragments used to induce antibodies to EMR2 have an amino acid sequence consisting of at least about 5 amino acids, and, more preferably, of at least about 14 ammo acids. It is also preferable that these oligopeptides, peptides, or fragments are identical to a portion of the amino acid sequence of the natural protein and contain the entire amino acid sequence of a small, naturally occurring molecule. Short stretches of EMR2 amino acids may be fused with those of another protein, such as KLH, and antibodies to the chimeric molecule may be produced.

Monoclonal antibodies to EMR2 may be prepared using any technique which provides for the production of antibody molecules by continuous cell lines in culture. These include, but are not limited to, the hybridoma technique, the human B-cell hybridoma technique, and the EBV-hybridoma technique (Kohler, G. et al. (1975) Nature 256:495-497; Kozbor, D. et al. (1985) J. Immunol. Methods 81:31-42; Cote, R. J. et al. (1983) Proc. Natl. Acad. Sci. 80:2026-2030; and Cole, S. P. et al. (1984) Mol. Cell Biol. 62:109-120, respectively).

In addition, techniques developed for the production of “chimeric antibodies,” such as the splicing of mouse antibody genes to human antibody genes to obtain a molecule with appropriate antigen specificity and biological activity, can be used (Morrison, S. L. et al. (1984) Proc. Natl. Acad. Sci. 81:6851-6855-Neuberger, M. S. et al. (1984) Nature 312:604-608; and Takeda, S. et al. (1985) Nature 314:452-454). Alternatively, techniques described for the production of single chain antibodies may be adapted, using methods known in the art, to produce EMR2-specific single chain antibodies. Antibodies with related specificity, but of distinct idiotypic composition, may be generated by chain shuffling from random combinatorial immunoglobulin libraries (Burton D. R. (1991) Proc. Nat. Acad. Sci. 88:10134-10137).

Antibodies may also be produced by inducing in vivo production in the lymphocyte population or by screening immunoglobulin libraries or panels of highly specific binding reagents as disclosed in the literature (Orlandi, R. et al. (1989) Proc. Natl. Acad. Sci. 86: 3833-3837; Winter, G. et al. (1991) Nature 349:293-299).

Antibody fragments which contain specific binding sites for EMR2 may also be generated. For example, such fragments include, but are not limited to, F(ab′)2 fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity (Huse, W. D. et al. (1989) Science 246:1275-1281).

Various immunoassays may be used for screening to identify antibodies having the desired specificity and minimal cross-reactivity. Numerous protocols for competitive binding or immunoradiometric assays using either polyclonal or monoclonal antibodies with established specificities are well known in the art. Such immunoassays typically involve the measurement of complex formation between EMR2 and its specific antibody. A two-site, monoclonal-based immunoassay utilising monoclonal antibodies reactive to two non-interfering EMR2 epitopes is preferred, but a competitive binding assay may also be employed (Maddox, supra).

Various methods such as Scatchard analysis in conjunction with radioimmunoassay techniques may be used to assess the affinity of antibodies for EMR2. Affinity is expressed as an association constant, Kₐ, which is defined as the molar concentration of EMR2-antibody complex divided by the molar concentrations of free antigen and free antibody under equilibrium conditions. The Kₐ determined for a preparation of polyclonal antibodies, which are heterogeneous in their affinities for multiple EMR2 epitopes, represents the average affinity, or avidity, of the antibodies for EMR2. The Kₐ determined for a preparation of monoclonal antibodies, which are monospecific for a particular EMR2 epitope, represents a true measure of affinity.

High-affinity antibody preparations with Kₐ, ranging from about 10⁹ to 10¹² L/mole are preferred for use in imnmunoassays in which the EMR2-antibody complex must withstand rigorous manipulations. Low-affinity antibody preparations with Kₐ, ranging from about 10⁶ to 10⁷ L/mole are preferred for use in immunopurification and similar procedures which ultimately require dissociation of EMR2, preferably in active form, from the antibody (Catty, D. (1988) Antibodies, Volume 1: A Practical Approach, IRL Press, Washington D.C.; Liddell,. J. E. and Cryer, A. (1991) A Practical Guide to Monoclonal Antibodies, John Wiley & Sons, New York N.Y.).

The titre and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is preferred for use in procedures requiring precipitation of EMR2-antibody complexes. Procedures for evaluating antibody specificity, titre, and avidity, and guidelines for antibody quality and usage in various applications, are generally available (Catty, supra; Coligan, supra).

The polypeptides of the invention or their fragments or analogues thereof, or cells expressing them, can also be used as immunogens to produce antibodies immunospecific for the EMR2 polypeptide polypeptides. The term “immunospecific” means that the antibodies have substantially greater affinity for the polypeptides of the invention than their affinity for other related polypeptides in the prior art.

Antibodies generated against the EMR2 polypeptide polypeptides can be obtained by administering the polypeptides or epitope-bearing fragments, analogues or cells to an animal preferably a non-human, using routine protocols. For preparation of monoclonal antibodies, any technique which provides antibodies produced by continuous cell line cultures can be used. Examples include the hybridoma technique (Kohler, G. and Milstein, C., Nature (1975) 256:495-497), the trioma technique, the human B-cell hybridoma technique (Kozbor et al, Immunology Today (1983) 4:72) and the EBV-hybridoma technique (Cole et al, MONOCLONAL ANTIBODIES AND CANCER THERAPY pp. 77-96. Alan R. Liss, Inc., 1985).

Techniques for the production of single chain antibodies (U.S. Pat. No. 4,946,778) can also be adapted to produce single chain antibodies to polypeptides of this invention. Also, transgenic mice, or other organisms including other mammals, may be used to express humanised antibodies.

The above-described antibodies may be employed to isolate or to identify clones expressing the polypeptide or to purify the polypeptides by affinity chromatography.

Antibodies against polypeptide polypeptides may also be employed to treat infections such as bacterial, fungal, protozoan and viral infections, particularly infections caused by HIV-1 or HIV-2; pain; cancers; anorexia; bulimia; asthma; Parkinson's disease; acute heart failure; hypotension; hypertension; urinary retention; osteoporosis; angina pectoris; myocardial infarction; ulcers; asthma; allergies; benign prostatic hypertrophy; and psychotic and neurological disorders, including anxiety, schizophrenia, manic depression, delirium, dementia, severe mental retardation and dyskinesias, such as Huntington's disease or Gilles dela Tourett's syndrome, among others.

The pharmaceutical compositions comprise a polypeptide, a polynucleotide, an antibody, an agonist or an antagonist of this invention, together with a pharmaceutically acceptable carrier or diluent.

In a further embodiment, a pharmaceutical composition comprising a substantially purified EMR2 polypeptide in conjunction with a suitable pharmaceutical carrier may be administered to a subject to treat or prevent a disorder associated with decreased expression or activity of EMR2 polypeptide including, but not limited to, those provided above.

In still another embodiment, an agonist which modulates the activity of EMR2 polypeptide maybe administered to a subject to treat or prevent a disorder associated with decreased expression or activity of EMR2 polypeptide including, but not limited to, those listed above.

In a further embodiment, an antagonist of EMR2 polypeptide may be administered to a subject to treat or prevent a disorder associated with increased expression or activity of EMR2 polypeptide. Such a disorder may include, but is not limited to, those respiratory, inflammatory, and immunological disorders discussed above. In one aspect an antibody which specifically binds EMR2 polypeptide may be used directly as an antagonist or indirectly as a targeting or delivery mechanism for bringing a pharmaceutical agent to cells or tissue which express EMR2 polypeptide.

In an additional embodiment, a vector expressing the complement of the polynucleotide encoding EMR2 polypeptide may be administered to a subject to treat or prevent a disorder associated with increased expression or activity of EMR2 polypeptide including, but not limited to, those described above.

In other embodiments, any of the proteins, antagonists, antibodies, agonists, complementary sequences, or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents way act synergistically to effect the treatment or prevention of the various disorders described above. Using this approach, one may be able to achieve therapeutic efficacy with lower dosages of each agent, thus reducing the potential for adverse side effects.

In another embodiment of the invention the polynucleotides encoding EMR2 polypeptide, or any fragment or complement thereof, may be used for therapeutic purposes. In one aspect, the complement of the polynucleotide encoding EMR2 polypeptide may be used in situations in which it would be desirable to block the transcription of the mRNA. In particular, cells may be transformed with sequences complementary to polynucleotides encoding EMR2 polypeptide. Thus, complementary molecules or fragments may be used to modulate EMR2 polypeptide activity, or to achieve regulation of gene function. Such technology is now well known in the art, and sense or antisense oligonucleotides or larger fragments can be designed from various locations along the coding or control regions of sequences encoding EMR2 polypeptide.

Expression vectors derived from retroviruses, adenoviruses, or herpes or vaccinia viruses, or from various bacterial plasmids, maybe used for delivery of nucleotide sequences to the targeted organ, tissue, or cell population. Methods which are well known to those skilled in the art can be used to construct vectors to express nucleic acid sequences complementary to the polynucleotides encoding EMR2 polypeptide (Ausubel, supra).

Genes encoding EMR2 polypeptide can be turned off by transforming a cell or tissue with expression vectors which express high levels of a polynucleotide, or fragment thereof, encoding EMR2 polypeptide. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector, and may last even longer if appropriate replication elements are part of the vector system.

Modifications of gene expression can be obtained by designing complementary sequences or antisense molecules (DNA, RNA, or PNA) to the control, 5′, or regulatory regions of the gene encoding the EMR2 polypeptide. Oligonucleotides derived from the transcription initiation site, e.g., between about positions −10 and +10 from the start site, are preferred. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al., (1994) in Huber, B. E. and B. I. Carr, Molecular and Immunologic Approaches, Futura Publishing Co., Mt. Kisco N.Y., pp. 163-177). A complementary sequence or antisense molecule may also be designed to block translation of mRNA by preventing the transcript from binding to ribosomes.

Ribozymes, enzymatic RNA molecules, may also be used to catalyst the specific cleavage of RNA. The mechanism of ribozyme action involves sequence-specific hybridisation of the ribozyme molecule to complementary target RNA, followed by endonucleolytic cleavage. For example, engineered hammerhead motif ribozyme molecules may specifically and efficiently catalyse endonucleolytic cleavage of sequences encoding EMR2 polypeptide.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may tender the oligonucleotide inoperable. The suitability of candidate targets may also be evaluated by testing accessibility to hybridisation with complementary oligonucleotides using ribonuclease protection assays.

Complementary ribonucleic acid molecules and ribozymes of the invention may be prepared by any method known in the art for the synthesis of nucleic acid molecules. These include techniques for chemically synthesising oligonucleotides such as solid phase phosphoramidite chemical synthesis. Alternatively, RNA molecules may be generated by in vitro and in vivo transcription of DNA sequences encoding EMR2 polypeptide. Such DNA sequences may be incorporated into a wide variety of vectors with suitable RNA polymerase promoters such as T7 or SP6. Alternatively, these CDNA constructs that synthesise complementary RNA, constitutively or inducibly, can be introduced into cell lines, cells, or tissues.

RNA molecules may be modified to increase intacellular stability and half-life. Possible modifications include, but are not limited to, the addition of flanking sequences at the 5′ and/or 3′ ends of the molecule, or the use of phosphorothioate or 2′O-methyl rather than phosphodiesterase linkages within the backbone of the molecule. This concept is inherent in the production of PNA's and can be extended in all of these molecules by the inclusion of non-traditional bases such as inosine, queosine, and wybutosine, as well as acetyl-, methyl-, thio-, and similarly modified forms of adenine, cytidine, guanine, thymine, and uridine which are not as easily recognised by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art (Goldman, C. K. et al. (1997) Nature Biotechnology 15:462-466).

Any of the therapeutic methods described above may be applied to any subject in need of such therapy, including, for example, mammals such as dogs, cats, cows, horses, rabbits, monkeys, and most preferably, humans.

An additional embodiment of the invention relates to the administration of a pharmaceutical or sterile conmposition, in conjunction with a pharmaceutically acceptable carrier for any of the therapeutic effects discussed above. Such pharmaceutical compositions may consist of EMR2 polypeptide, antibodies to EMR2 polypeptide, and mimetics, agonists, antagonists, or inhibitors of EMR2 polypeptide. The compositions may be administered alone or in combination with at least one other agent, such as a stabilising compound, which may be administered in any sterile, biocompatible pharmaceutical carrier including, but not limited to, saline, buffered saline, dextrose, and water. The compositions may be administer to a patient alone, or in combination with other agents, drags, or hormones.

The pharmaceutical compositions utilised in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combining active compounds with solid excipient and processing the resultant mix of granules (optionaly, after grinding) to obtain tablets or dragee cores. Suitable auxiliaries can be added, if desired. Suitable excipients include carbohydrate or protein fillers, such as sugars, including lactose, sucrose, mannitol, and sorbitol; starch from corn, wheat, rice, potato, or other plants; cellulose, such as methyl cellulose, hydroxypropylmethyl-cellulose, or sodium carboxymethylcellulose; gums, including arabic and tragacanth; and proteins, such as gelatin and collagen. If desired, disintegrating or solubilising agents may be added, such as the cross-linked polyvinyl pyrrolidone, agar, and alginic acid or a salt thereof; such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterise the quantity of active compound, i.e., dosage.

Phamaceutical preparations which can be used orally include push-fit capsules made of gelatin, as well as soft, scaled capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilisers. In soft capsules, the active compounds may be dissolved or suspended in suitable liquids, such as fatty oils, liquid, or liquid polyethylene glycol with or without stabilisers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilisers or agents to increase the solubility of the compounds and allow for the preparation of highly concentrated solutions.

For topical or nasal administration, penetrants appropriate to the particlar barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilising processes.

The pharmaceutical composition may be provided as a salt and can be formed with many acids, including but not limited to, hydrochloric, sulphuric, acetic, lactic, tartaric, malic, and succinic acid. Salts tend to be more soluble in aqueous or other protonic solvents than are the corresponding free base forms. In other cases, the preferred preparation may be a lyophilised powder which may contain any or all of the following: 1 mM to 50 mM histidine, 0.1% to 2% sucrose, and 2% to 7% mannitol, at a pH range of 4.5 to 5.5, that is combinaed with buffer prior to use.

After pharmaceutical compositions have been prepared, they can be placed in an appropriate container and labelled for treatment of an indicated condition. For administration of EMR2 polypeptide, such labelling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells or in animal models such as mice, rats, rabbits, dogs, or pigs. A animal model may also be used to determine the appropriate concentration range and route of administration. Such information can then be used to determine useful doses and routes for administration in humans.

A therapeutically effective dose refers to that amount of active ingredient, for example EMR2 polypeptide or fragments thereof, antibodies of EMR2 polypeptide, and agonists, antagonists or inhibitors of EMR2 polypeptide, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of therapeutic to toxic effects is the thereutic index, and it can be expressed as the ED₅₀/LD₅₀ ratio. Pharmaceutical compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general heath of the subject, the age, weight, and gender of the subject, time and frequency of administration, drug combination(s), reaction sensitivities, and response to therapy. Long-acting pharmaceutical compositions may be administered every 3 to 4 days, every week, or bi-weekly depending on the half-life and clearance rate of the particular formulation.

Normal dosage amounts may vary from about 0.1 μg to 100,00 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

It will be appreciated that this invention provides methods of treating an abnormal conditions related to both an excess of and insufficient amounts of EMR2 polypeptide activity.

If the activity of EMR2 polypeptide is in excess, several approaches are available. One approach comprises administering to a subject an inhibitor compound (antagonist) along with a pharmaceutically acceptable carrier in an amount effective to inhibit activation by blocking binding of ligands to the EMR2 polypeptide, or by inhibiting a second sign, and thereby alleviating the abnormal condition.

In another approach, soluble forms of EMR2 polypeptides still capable of binding the ligand in competition with endogenous EMR2 polypeptide may be administered. Typical embodiments of such competitors comprise fragments of the EMR2 polypeptide.

In still another approach, expression of the gene encoding endogenous EMR2 polypeptide can be inhibited using expression blocking techniques. Known such techniques involve the use of antisense sequences, either internally generated or separately administered. See, for example, O'Connor. J. Neurochem. (1991) 56:560 in Oligodeoxynucleotides as Antisense Inhibitors of Gene Expression, CRC Press, Boca Raton, Fla. (1968). Alternatively, oligonucleotides which form triple helices with the gene can be supplied. See, for example, Lee et al, Nucleic Acids Res. (1979) 6:3073; Cooney et al., Science (1988) 241:456; Dervan et al., Science (1991) 251:1360. These oligomers can be administered per se or the relevant oligomers can be expressed in vivo.

For treating abnormal conditions related to an under-expression of EMR2 polypeptide and its activity, several approaches are also available. One approach comprises administering to a subject a therapeutically effective amount of a compound which activates EMR2 polypeptide, i.e., an agonist as described above, in combination with a pharmaceutically acceptable carrier to thereby alleviate the abnormal condition. Alternatively, gene therapy may be employed to effect the endogenous production of EMR2 polypeptide by the relevant cells in the subject. For example, a polynucleotide of the invention maybe engineered for expression in a replication defective retroviral vector, as discussed above. The retroviral expression construct may then be isolated and introduced into a packaging cell transduced with a retroviral plasmid vector containing RNA encoding a polypeptide of the present invention, such that the packaging cell now produces infectious viral particles containing the gene of interest. These producer cells may be administered to a subject for engineering cells in vivo and expression of the polypeptide in vivo. For overview of gene therapy see Chapter 20, Gene Therapy and other Molecular Genetic-based Therapeutic Approaches, (and references cited therein) in Human Molecular Genetics, T. Strachan and A. P Read, BIOS Scientific Publishers Ltd (1996).

Peptides, such as the soluble form of EMR2 polypeptides, and agonists and antagonist peptides or small molecules, may be formulated in combination with a suitable pharmaceutical carrier, such as discussed above. Such formulations comprise a therapeutically effective amount of the polypeptide or compound, and a pharmaceutically acceptable carier or excipient. Such carriers include but are not limited to, saline, buffered saline, dextrose, water, glycerol, ethanol, and combinations thereof. Formulation should suit the mode of administration, and is well within the skill of the art. The invention further relates to pharmaceutical packs and kits comprising one or more containers filled with one or more of the ingredients of the aforementioned compositions of the invention.

Polypeptides and other compounds of the present invention may be employed alone or in conjunction with other compounds, such as therapeutic compounds.

Preferred forms of systemic administration of the pharmaceutical compositions include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if properly formulated in enteric or encapsulated formulations, oral administration may also be possible. Administration of these compounds may also be topical and/or localised, in the form of salves, pastes, gels and the like.

Wide variations in the needed dosage are to be expected in view of the variety of compounds available and the differing efficiencies of various routes of administration. For example, oral administration would be expected to require higher dosages then administration by intravenous injection. Variations in these dosage levels can be adjusted using standard empirical routines for optimisation, as is well understood in the art.

Polypeptides used in treatment can be generated endogenously in the subject, in treatment modalities often referred to as “gene therapy”, such as described above. Thus, for example, cells from a subject may be engineered with a polynucleotide, such as a DNA or RNA, to encode a polypeptide ex vivo, and for example, by the use of a retroviral plasmid vector. The cells are then introduced into the subject.

The invention also provides a diagnostic method involving assessing expression of EMR2 protein or involving identification of a mutation in DNA encoding such protein. The invention further provides a diagnostic method involving use of a polynucleotide of this invention as a probe. The probe can be employed with cells or other samples which might express EMR2 protein.

This invention also relates to the use of EMR2 polynucleotides for use as diagnostic reagents. Detection of a mutated form of the EMR2 gene associated with a dysfunction will provide a diagnostic tool that can add to or define a diagnosis of a disease or susceptibility to a disease which results from under-expression, over-expression or altered expression of EMR2 polypeptide. Individuals carrying mutations in the EMR2 gene may be detected at the DNA level by a variety of techniques.

Nucleic acids for diagnosis maybe obtained from a subject's cells, such as from blood, urine, saliva, tissue biopsy or autopsy material. The genomic DNA may be used directly for detection or may be amplified enzymatically by using PCR or other amplification techniques prior to analysis. RNA or CDNA may also be used in similar fashion. Deletions and insertions can be detected by a change in size of the amplified product in comparison to the normal genotype. Point mutations can be identified by hybridising amplified DNA to labelled EMR2 polypeptide nucleotide sequences. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing. See, e.g., Myers et al, Science (1985) 230:1242. Sequence changes at specific locations may also be revealed by nuclease protection assays, such as RNase and S1 protection or the chemical cleavage method. See Cotton et al., Proc Natl Acad. Sci USA (1985) 85:4397-4401.

In another embodiment, an array of oligonucleotide probes comprising the EMR2 polypeptide nucleotide sequence or fragments thereof can be constructed to conduct efficient screening of e.g., genetic mutations. Array technology methods are well known and have general applicability and can be used to address a variety of questions in molecular genetics including gene expression, genetic linkage, and genetic variability. (See for example: M. Chee et al., Science, Vol 274, pp 610-613 (1996)).

The diagnostic assays offer a process for diagnosing or determining a susceptibility to infections such as bacterial, fungal, protozoan and viral infections, particularly infections caused by HIV-1 or HIV-2; pain; cancers; anorexia; bulimia; Parkinson's disease; acute heart failure; hypotension; hypertension; urinary retention; osteoporosis; angina pectoris; myocardial infraction; ulcers; asthma; allergies; benign prostatic hypertrophy, and psychotic and neurological disorders, including anxiety schizophrenia, manic depression, delirium, dementia, severe mental retardation and dyskinesias, such as Huntington's disease or Gilles de la Tourett's syndrome through detection of mutation in the EMR2 polypeptide gene by the methods described.

In addition, such conditions can be diagnosed by methods comprising determining from a sample derived from a subject an abnormally decreased or increased level of the EMR2 polypeptide or EMR2 mRNA. Decreased or increased expression can be measured at the RNA level using any of the methods well known in the art for the quantitation of polynucleotides, such as, for example, PCR, RT-PCR, RNase protection, Northern blotting and other hybridisation methods. Assay techniques that can be used to determine levels of a protein, such as an EMR2 polypeptide, in a sample derived from a host are well-known to those of skill in the art. Such assay methods include radioimmunoassays, competitive-binding assays, Western Blot analysis and ELISA assays.

Thus in another aspect, the present invention relates to a diagnostic kit for a disease or susceptibility to a disease, particularly infections such as bacterial, fungal, protozoan and viral infections, particularly infections caused by HIV-1 or HIV-2; pain; cancers; anorexia; bulimia; asthma; Parkinson's disease; acute heart failure; hypotension; hypertension; urinary retention; osteoporosis; angina pectoris; myocardial infarction; ulcers; asthma; allergies; benign prostatic hypertrophy; and psychotic and neurological disorders, including anxiety, schizophrenia, manic depression, delirium, dementia, severe mental retardation and dyskinesias, such as Huntington's disease or Gilles dela Tourett's syndrome, which comprises:

(a) an EMR2 polynucleotide, preferably the nucleotide sequence of SEQ ID NO: 1, or a fragment thereof;

(b) a nucleotide sequence complementary to that of (a);

(c) a EMR2 polypeptide, preferably the polypeptide of SEQ ID NO: 2, or a fragment thereof; or

(d) an antibody to an EMR2 polypeptide, preferably to the polypeptide of SEQ ID NO: 2.

It will be appreciated that in any such kit, (a), (b), (c) or (d) may comprise a substantial component.

The nucleotide sequences of the present invention are also valuable for chromosome identification. The sequence is specifically targeted to and can hybridise with a particular location on an individual human chromosome. The mapping of relevant sequences to chromosomes according to the present invention is an important first step in correlating those sequences with gene associated disease. Once a sequence has been mapped to a precise chromosomal location, the physical position of the sequence on the chromosome can be correlated with genetic map data. Such data are found, for example, in V. McKusick, Mendelian Inheritance in Man (available on line through Johns Hopkins University Welch Medical Library). The relationship between genes and diseases that have been mapped to the same chromosomal region are then identified through linkage analysis (coinheritance of physically adjacent genes).

The differences in the CDNA or genomic sequence between affected and unaffected individuals can also be determined. If a mutation is observed in some or all of the affected individuals but not in any normal individuals, then the mutation is likely to be the causative agent of the disease.

In another embodiment antibodies which specifically bind EMR2 polypeptide may be used for the diagnosis of disorders characterised by expression of EMR2 polypeptide, or in assays to monitor patients being treated with polypeptide or agonists, antagonists, or inhibitors of EMR2 polypeptide. Antibodies useful for diagnostic purposes may be prepared in the same manner as described above for therapeutics. Diagnostic assays for EMR2 polypeptide include methods which utilise the antibody and a label to detect EMR2 polypeptide in human body fluids or in extracts of cells or tissues. The antibodies may be used with or without modification, and may be labelled by covalent or non-covalent attachment of a reporter molecule. A wide variety of reporter molecules, several of which are described above, are known it the art and may be used.

A variety of protocols for measuring EMR2 polypeptide, including ELISA's, RIA's, and FAC's, are known in the art and provide a basis for diagnosing altered or abnormal levels of EMR2 polypeptide expression. Normal or standard values for EMR2 polypeptide expression are established by combining body fluids or cell extracts taken from normal mammalian subjects, preferably human, with antibody to EMR2 polypeptide under conditions suitable for complex formation. The amount of standard complex formation may be quantitated by various methods, preferably by photometric means. Quantities of EMR2 polypeptide expressed in subject, control, and disease samples from biopsied tissues are compared with the standard values. Deviation between standard and subject values establishes the parameters for diagnosing disease.

As noted above, the polynucleotides encoding EMR2 polypeptide may be used for diagnostic purposes. The polynucleotides which may be used include oligonucleotide sequences, complementary RNA and DNA molecules, and PNA's. The polynucleotides may be used to detect and quantitate gene expression in biopsied tissues in which expression of EMR2 polypeptide may be correlated with disease. The diagnostic assay may be used to determine absence, presence, and excess expression of EMR2 polypeptide, and to monitor regulation of EMR2 polypeptide levels during therapeutic intervention.

In one aspect, hybridisation with PCR probes which are capable of detecting polynucleotide sequences, including genomic sequences, encoding EMR2 polypeptide or closely related molecules may be used to identify nucleic acid sequences which encode EMR2 polypeptide. The specificity of the probe, whether it is made from a highly specific region, e.g., the 5′ regulatory region, or from a less specific region, e.g., a conserved motif, and the stringency of the hybridisation or amplification (maximal, high, intermediate, or low), will determine whether the probe identifies only naturally occurring sequences encoding EMR2 polypeptide, allelic variants, or related sequences.

Probes may also be used for the detection of related sequences, and should preferably have at least 50% sequence identity to any of the EMR2 polypeptide encoding sequences. The hybridisation probes of the subject invention may be DNA or RNA and may be derived from the sequence of SEQ ID NO: 2 or from genomic sequences including promoters, enhancers, and introns of the EMR2 polypeptide gene.

Means for producing specific hybridisation probes for DNA's encoding EMR2 polypeptide include the cloning of polynucleotide sequences encoding EMR2 polypeptide or EMR2 polypeptide derivatives into vectors for the production of mRNA probes. Such vectors are known in the art, are commercially available, and may be used to synthesise RNA probes in vitro by means of the addition of the appropriate RNA polymerases and the appropriate labelled nucleotides. Hybridisation probes may be labelled by a variety of reporter groups, for example, by radionuclides such as ³²P or ³⁵S, or by enzymatic labels, such as alkaline phosphate coupled to the probe via avidin/biotin coupling systems, and the like.

Polynucleotide sequences encoding EMR2 polypeptide may be used for the diagnosis of a disorder associated with expression of EMR2 polypeptide. Examples of such a disorder include, but are not limited to, a respiratory disorder, such as, allergies, asthma, acute inflammatory lung disease, chronic inflammatory lung disease, chronic obstructive pulmonary dysplasia emphysema, adult respiratory distress syndrome, bronchitis, and intestinal lung diseases; complications of cancer, haemodialysis, and extracorporeal circulation, viral, bacterial, fungal, parasitic, protozoal and helminthic infections, and trauma; an inflammatory disorder, such as rheumatoid arthritis, osteoarthritis, inflammatory bowel disease, adult respiratory distress syndrome, allergies, asthma, bronchitis, cholecystitis, contact dermatitis, Crohn's disease, atopic dermatitis, emphysema, episodic lymphopaenia with lymphocytotoxins, erythroblastosis fetalis, erythema nodosum, atrophic gastritis, glomerulonephritis, Goodpasture's syndrome, gout Graves' disease, Hashimoto's thyroiditis, hypereosinophilia, interstitial lung disease, myasthenia gravis, myocardial or pericardial inflammation, pancreatitis, polymyositis, psoriasis, Reiter's syndrome, scleroderma, Sjögren's syndrome, systemic anaphylaxis, systemic sclerosis, thrombocytopenic purpura, ulcerative colitis, uveitis, Werner syndrome, haemodialysis, and extracorporeal circulation, and trauma; and an immunological disorder, such as, acquired immunodeficiency syndrome (AIDS), Addison's disease, adult respiratory distress syndrome, allergies, ankylosing spondylitis, amyloidosis, anaemia, atherosclerosis, autoimmune haemolytic anaemia, autoimmune thyroiditis, dermatomyositis, diabetes mellitus, irritable bowel syndrome and multiple sclerosis.

The polynucleotide sequences encoding EMR2 polypeptide may be used in Southern or Northern analysis, dot blot, or other membrane-based technologies; in PCR technologies; in dipstick, pin, and ELISA assays; and in microarrays utilising fluids or tissues from patients to detect altered EMR2 polypeptide expression. Such qualitative or quantitative methods are well known in the art.

In a particular aspect, the nucleotide sequences encoding EMR2 polypeptide may be useful in assays tat detect the presence of associated disorders, particularly those mentioned above. The nucleotide sequences encoding EMR2 polypeptide may be labelled by standard methods and added to a fluid or tissue sample from a patient under conditions suitable for the formation of hybridisation complexes. After a suitable incubation period, the sample is washed and the signal is quantitated and compared with a standard value. If the amount of signal in the patient sample is significantly altered in comparison to a control sample then the presence of altered levels of nucleotide sequences encoding EMR2 polypeptide in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of EMR2 polypeptide, a normal or standard profile for expression is established. This may be accomplished by combining body fluids or cell exacts taken from normal subjects, either animal or human, with a sequence, or a fragment thereof, encoding EMR2 polypeptide, under conditions suitable for hybridisation or amplification. Standard hybridisation may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridisation assays may be repeated on a regular basis to determine if the level of expression in the patient begins to approximate that which is observed in the normal subject. The results obtained from successive assays maybe used to show the efficacy of treatment over a period ranging from several days to months.

With respect to cancer, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Additional diagnostic uses for oligonucleotides designed from the sequences encoding EMR2 polypeptide may involve the use of PCR. These oligomers may be chemically synthesised, generated enzymatically, or produced in vitro. Oligomers will preferably contain a fragment of a polynucleotide encoding EMR2 polypeptide, or a fragment of a polynucleotide complementary to the polynucleotide encoding EMR2 polypeptide, and will be employed under optimised conditions for identification of a specific gene or condition. Oligomers may also be employed under less stringent conditions for detection or quantitation of closely related DNA or RNA sequences.

Methods which may also be used to quantitate the expression of EMR2 polypeptide include radiolabelling or biotinylatng nucleotides, co-amplification of a control nucleic acid, and interpolating results from standard curves (Melby, P. C. et al. (1993) J. Immunol. Methods 159:235-244; Duplaa, C. et al. (1993) Anal. Biochem. 229-236). The speed of quantitation of multiple samples may be accelerated by running the assay in an ELISA format where the oligomer of interest is presented in various dilutions and a spectrophotometric or calorimetric response gives rapid quantitation.

In further embodiments, oligonucleotides or longer fragments derived from any of the polynucleotide sequences described herein may be used as targets in a microarray. The microarray can be used to monitor the expression level of large numbers of genes simultaneously and to identify genetic variants, mutations, and polymorphisms. This information may be used to determine gene function, to understand the genetic basis of a disorder, to diagnose a disorder, and to develop and monitor the activities of therapeutic agents.

Microarrays may be prepared, used, and analysed using methods known in the art. See, for example, Brennan, T. M. et al. (1995) U.S. Pat. No. 5,474,796; Schena, M. et al. (I 996) Proc. Natl. Acad. Sci. 93:10614-10619; Baideschweiler et al. (I 995) PCT application W095/251116; Shalon, D. et al. (1995) PCT application WO95/35505; Heller, R. A. et al. (1997) Proc. Nat. Acad. Sci. 94:2150-2155; and Heller, M. J. et al. (1997) U.S. Pat No. 5,605,662.

In another embodiment of the invention, nucleic acid sequences encoding EMR2 polypeptide may be used to generate hybridisation probes useful in mapping the naturally occurring, genomic sequence. The sequences may be mapped to a particular chromosome, to a specific region of a chromosome, or to artificial chromosome constructions, e.g., human artificial chromosomes (HAC's), yeast artificial chromosomes (YAC's), bacterial artificial chromosomes (BAC's), bacterial PI constructions, or single chromosome CDNA libraries (Price, C. M. (1993) Blood Rev. 7:127-134; Trask, B. J. (1991) Trends Genet. 7:149-154).

Fluorescent in situ hybridisation (FISH) may be correlated with other physical chromosome mapping techniques and genetic map data (Heinz-Ulrich, et al. (1995) in Meyers, R. A. (ed.) Molecular Biology and Biotechnology, VCH Publishers New York N.Y., pp. 965-968). Examples of genetic map data can be found in various scientific journals or at the Online Mendelian Inheritance in Man (OMIM) site. Correlation between the location of the gene encoding EMR2 polypeptide on a physical chromosomal map and a specific disorder, or a predisposition to a specific disorder, may help define the region of DNA associated with that disorder. The nucleotide sequences of the invention may be used to detect differences in gene sequences among normal, carrier, and affected individuals.

In situ hybridisation of chromosomal preparations and physical mapping techniques, such as linkage analysis using established chromosomal markers, may be used for extending genetic maps. Often the placement of a gene on the chromosome of another mammalian species, such as mouse, may reveal associated markers even if the number or arm of a particular human chromosome is not known. New sequences can be assigned to chromosomal arms by physical mapping. This provides valuable information to investigators searching for disease genes using positional cloning or other gene discovery techniques. Once the disease or syndrome has been crudely localised by genetic linkage to a particular genomic region, e.g., ataxia-telangiectasia to I11q22-23, any sequences mapping to that area may represent associated or regulatory genes for further investigation (Gatti, R. A. et al. (1988) Nature 336:577-580). The nucleotide sequence of the subject invention may also be used to detect differences in the chromosomal location due to translocation, inversion, etc., among normal, carrier, or affected individuals.

In another embodiment of the invention, EMR2 polypeptide, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between EMR2 polypeptide and the agent being tested may be measured.

Another technique for drug screening provides for high throughput screening of compounds having suitable binding affinity to the protein of interest (Geysen, et al. (1984) PCT application W084/03564). In this method, large numbers of different small test compounds are synthesised on a solid substrate, such as plastic pins or some other surface. The test compounds are reacted with EMR2 polypeptide, or fragments thereof, and washed. Bound EMR2 polypeptide is then detected by methods well known in the art. Purified EMR2 polypeptide can also be coated directly onto plates for use in the aforementioned drug screening techniques. Alternatively, non-neutralising antibodies can be used to capture the peptide and immobilise it on a solid support.

In another embodiment, one may use competitive drug screening assays in which neutralising antibodies capable of binding EMR2 polypeptide specifically compete with a test compound for binding EMR2 polypeptide. In this manner, antibodies can be used to detect the presence of any peptide which shares one or more antigenic determinants with EMR2 polypeptide.

In additional embodiments, the nucleotide sequences which encode EMR2 polypeptide may be used in any molecular biology techniques that have yet to be developed, provided the new techniques rely on properties of nucleotide sequences that are currently known, including, but not limited to, such properties as the triplet genetic code and specific base pair interactions.

A method of treatment in accordance with this invention involves administering as appropriate a polypeptide, a polynucleotide, an antibody, an agonist or an antagonist of this invention.

Additionally, the present invention is founded on the discovery of an observed function of EMR2, in cell-cell adhesion. This adhesion is primarily neutrophil-macrophage binding, and as such the present invention can be employed for treatment of conditions which involve such binding.

### BRIEF DESCRIPTION OF THE DRAWINGS

FIG. 1 shows the full-length cDNA sequence and deduced amino acid sequence of human EMR2;

FIG. 2 shows the amino acid sequence alignments of EMR2, CD97 and EMR1;

FIG. 3 shows the location and structure of the EMR2 gene on human chromosome 19p13.1;

FIG. 4 shows the tissue and cell-type expression patterns of EMR2;

FIG. 5 shows the alternative splicing of EMR2 RNA, transcripts;

FIG. 6 shows the cell surface expression of EMR2 on transfected cells;

FIG. 7 shows that EMR2 does not interact with CD55;

FIG. 8 is a schematic representation of the multimeric protein probes used for ligand search;

FIG. 9 demonstrates that EMR2(EGF-1,2,3,4,5)-fluorescent beads bind to a putative EMR2 ligand on monocyte-derived macrophages;

FIG. 10 is a schematic representation of a possible interaction of EMR2 on neutrophils and its putative ligand on macrophages;

FIG. 11 shows that 2A1 mAb specifically recognised EMR2;

FIG. 12 shows FACS analysis of EMR2 beads;

FIG. 13 shows tissue staining by the EMR2 protein-fluorescent bead complex; and

FIG. 14 shows tissue staining patterns of the EMR2-ligand.

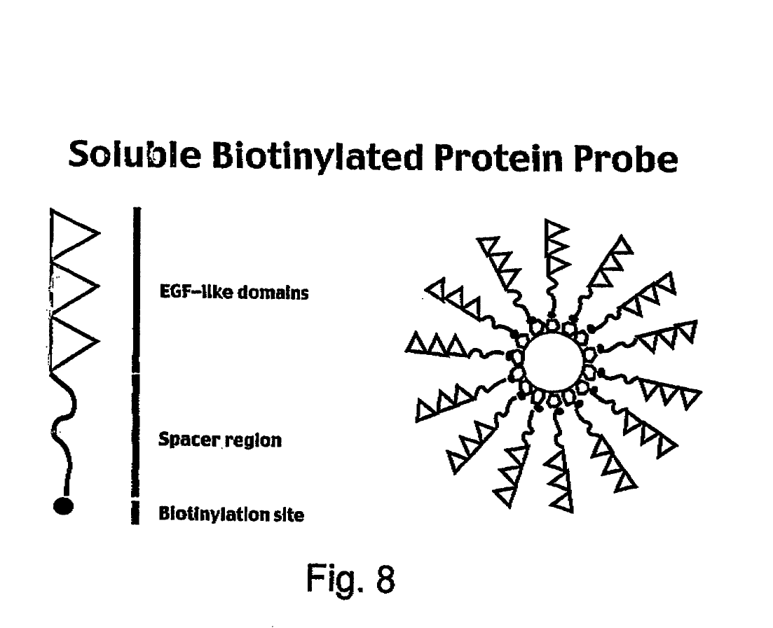

The following FIGS. 8, 9 and 10 relate to our discovery of a function for the EMR2 protein.

FIG. 8 is a schematic representation of the multimeric protein probes used for ligand search. FIG. 8(I) shows the soluble recombinant biotinylated EMR2 protein, while FIG. 8(ii) shows the biotinylated EMR2 protein-fluorescent bead complex. Biotinylated EMR2 proteins are coupled to the beads via avidin.

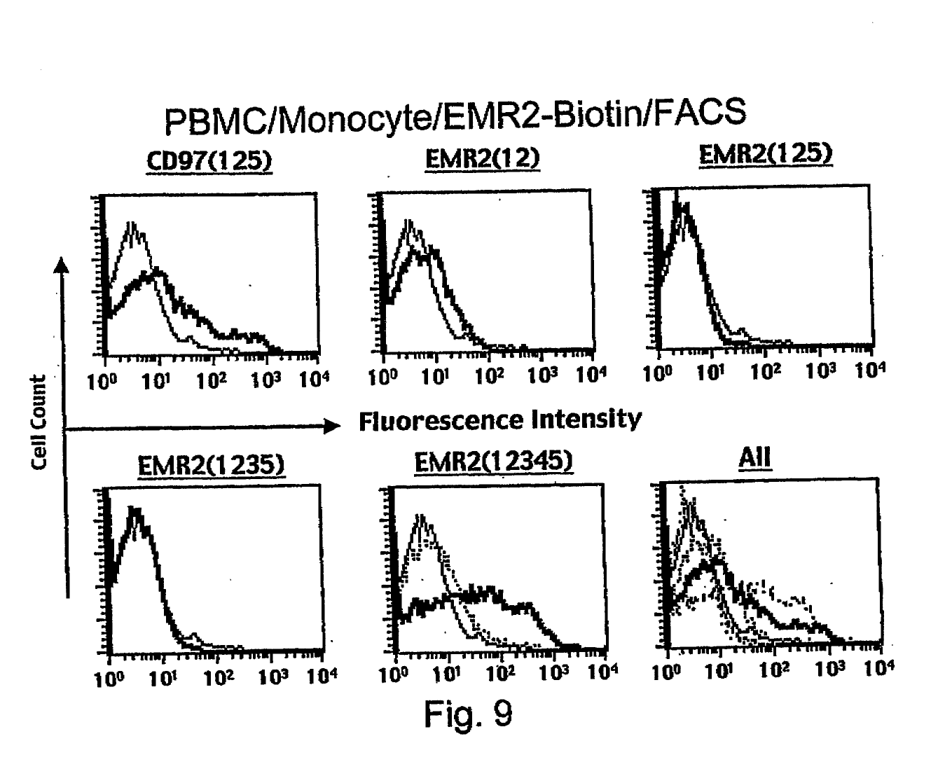

FIG. 9 demonstrates that EMR2(EGF-1,2,3,4,5)-fluorescent beads bind to a putative EMR2 ligand on monocyte-derived macrophages. Diagrams show FACS analysis of the interaction of various biotinylated EMR2 protein isoforms-fluorescent beads with human monocyte-derived macrophages. EMR2(EGF1, 2, 3, 4, 5)-fluorescent beads bind to 45-50% of the cells and the interaction is abolished in the presence of EGTA (dash line). CD97(EGF-1, 2, 5)-fluorescent beads binding to CD55 was used as a possible control.

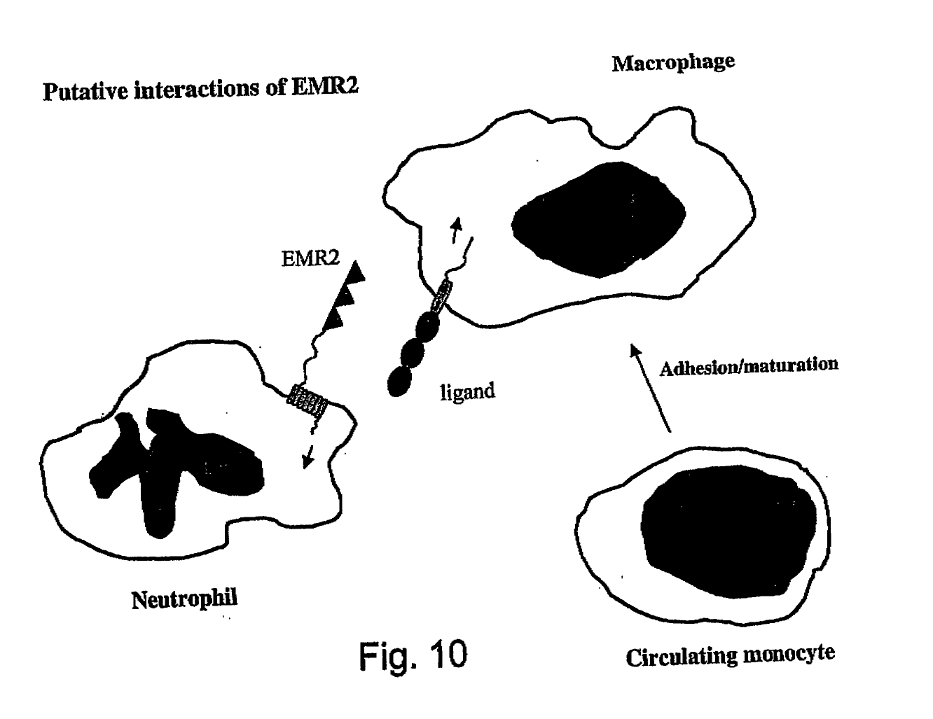

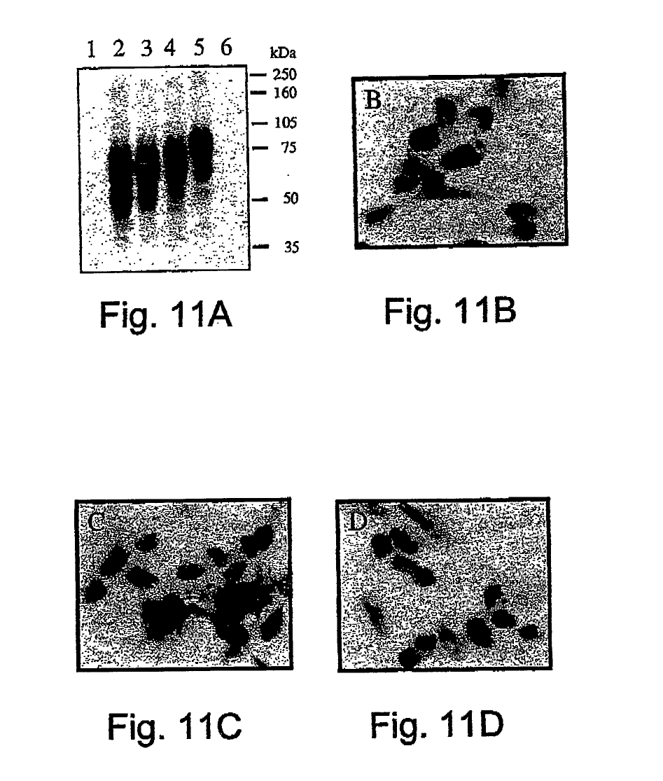

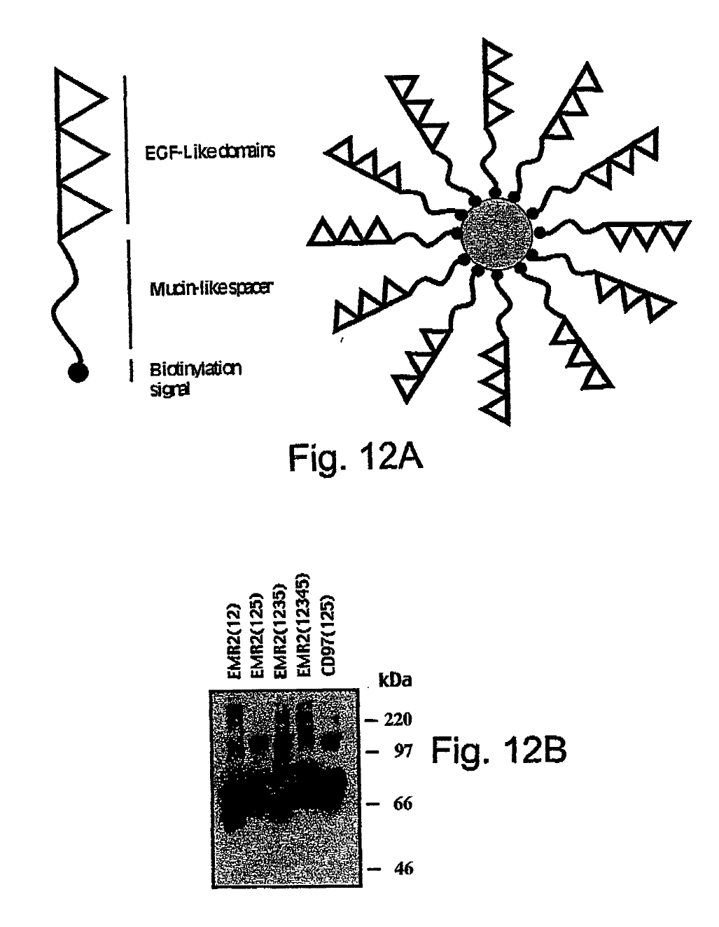

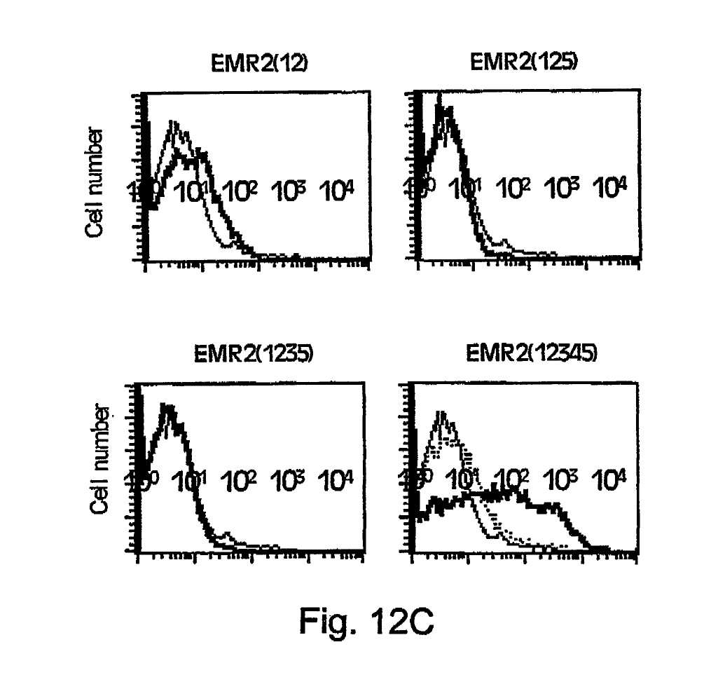

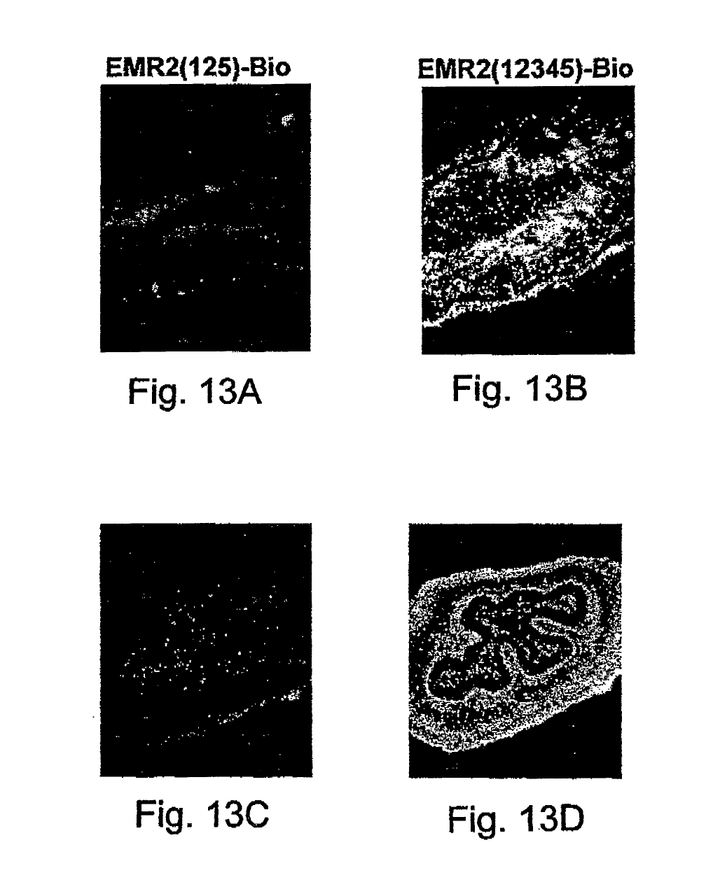

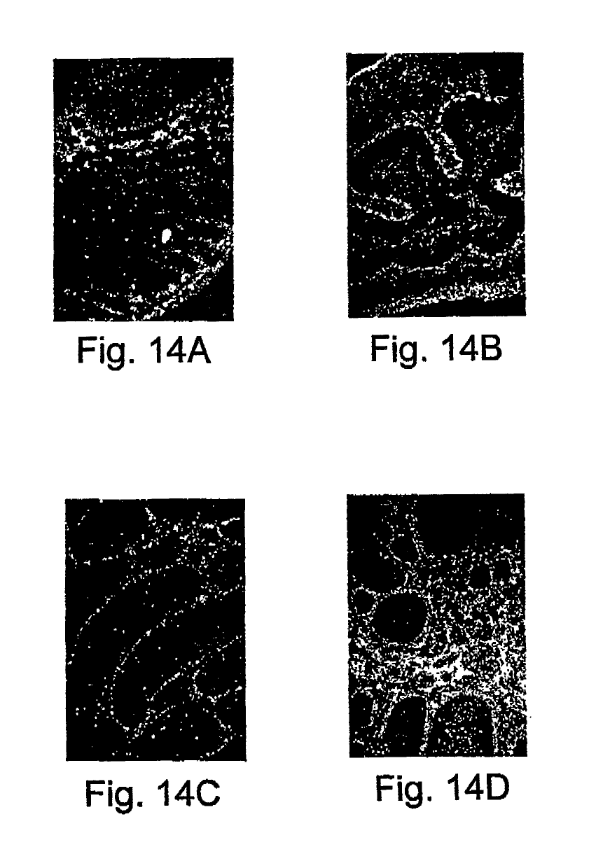

FIG. 10 is a schematic representation of a possible interaction of EMR2 on neutrophils and its putative ligand on macrophages. Cross talk EMR2 and the putative ligand leading to possible intracellular signalling is also presented.

The abbreviations used herein are: aa, amino acid; antigen, Ag; bp, base pair; EGF, epidermal growth factor; EMR, EGF-like module containing mucin-like hormone receptor; FCS, foetal calf serum; FACS, fluorescence-activated cell sorting; GPCR, G protein-coupled receptor; Mφ, macrophage; mAb, monoclonal antibody; nt, nucleotide; ORF, open reading frame; PBMC, peripheral blood monocytic cells; PMN, polymorphonuclear cells; RACE, rapid amplification of cDNA ends; RT-PCR, reverse transcription polymerase chain reaction.

### EXAMPLE

Materials and Methods

Materials-All chemicals and reagents were obtained from Sigma unless otherwise specified. Cell culture media and supplements were purchased from Life Technologies Inc. X-Vivo 10 medium was from BioWhittaker (Walkersville, Md.). Human buffs coats (negative for HIV and Hepatitis B) were purchased from the National Blood Service, Bristol, U.K. Cell lines were provided by the cell bank at the Sir William Dunn School of Pathology, University of Oxford.

Database Search and cDNA Cloning of the EMR2 Gene-The cDNA sequences of the human CD97, human EMR1 and mouse Emrl/F4/80 genes (GenBank accession numbers X84700, X81479 and U66888, respectively) were used to search for homologous DNA sequences in the GenBank/EMBL databases using the BLAST algorithm. Two overlapping human cosmids, R29368 and F21185 (GenBank accession numbers AC004262 and AC005327), were found to contain a putative coding sequence (designated EMR2) highly homologous to the EMR1 and CD97 genes. To characterise the full-length EMR2 cDNA sequence, DNA fragments were amplified from a human spleen Marathon Ready cDNA library (CLONTECH) by Rapid Amplification of cDNA Ends (RACE)-polymerase chain reaction (PCR) under conditions recommended by the manufacturer (CLONTECH). Two rounds of PCR reactions were conducted to amplify visible EMR2 cDNA fragments. Marathon adapter primers, Ap-1 (5′-CCATCCTAATACGACTCACTATAGGGC) (SEQ ID NO. 3) and AP-2 (5′-ACTCACTATAGGGCTCGAGCGGC) (SEQ ID NO. 4) were paired individually with nested EMR2-specific primers, 5′-1 (5′-CTGGTGTTCTGGATGGCTTTACACAGGAG) (SEQ ID NO. 5) and 5′-2 (5′-TGCACATCGTAGTGGGCCATGA) (SEQ ID NO. 6) as well as 3′-1 (5′-AGGTGCTCTGTGTCTTCTCA) (SEQ ID NO. 7) and 3′-2 (5′-GTGCTGTGCTCCATCATCGCCG) (SEQ ID NO. 8) to generate 5′-RACE and 3′-RACE fragments, respectively. EMR2 cDNA fragments were separated on a 1% agarose gel, purifed, subcloned and sequenced using standard molecular techniques. In an attempt to isolate cDNA clones containing the full-length EMR2 open reading fame (ORF), a human spleen RAPID SCREEN™ cDNA Libary panel (OriGene Technologies, Inc) was screened by a 96-well PCRformat using two EMR2 sequence-specific primers (5′-TTCCACCGGCAAAGAGGGAAGATCTT (SEQ ID NO. 9) and 5′-GACAGTGGCCATTTCTGCAGCCTCCAG (SEQ ID NO. 10)). Three individual positive cDNA clones were identified. Restriction enzyme digestion analysis and DNA sequencing revealed that these are overlapping clones with sequence identity to the RACE-PCR cDNA clones.

Sequence Analysis-DNA sequencing reactions were performed using the BigDye™ Terminator DNA sequencing kit (PE Applied Biosystems). Samples were electrophoresed on an ABI 373A DNA Sequencer and analysed by ABI Prism Model Version 2.1.1 software (PE Applied Biosystems). Homologous DNA sequence search was carried out using BLAST algorithm against DNA sequences in GenBank/EMBL databases. Protein alignment, alignment of consensus sequences and the percent identity were analysed by the Pileup, the Prettybox, and the Gap program, respectively.

Cell Culture-All the culture media were supplemented with 10% heat inactivated foetal calf serum (FCS), 2 mM L-glutamine, 50 IU/ml penicillin and 50 μ/ml streptomycin. Cell lines U937, K562, Monomac6, HL60, HUT-78, Jurkat, H9, RAJI and Daudi were cultured in RPMI1640 medium. HepG2 and HEK293 cells were grown in Eagle's minimum essential medium (EMEM). CHO-K1 cells were cultured in Ham's F-12 medium. COS-7 cells were maintained in Dulbecco's modified Eagle's medium (DMEM). All cells were incubated at 37 C. in a 5% Co₂, 95% humidity incubator. Human blood monocytes/Mφs were prepared from peripheral blood mononuclear cells (PBMC's) by adherence as previously described (Herbein et al., 1995). The adherent monocytes were cultured in X-Vivo 10 medium supplemented with 1% autologous serum for up to 7 days. In some cases, PBMC's were subjected to two cycles of adherence to prepare strongly adherent monocytes (the first cycle) and weakly adherent monocytes (the second cycle) for RNA analysis. Human polymorphonuclear cells (PMN's)/granulocytes were isolated from healthy donors' heparinised whole blood using Polymorphprep™ (NYCOMED PHARMA AS, Oslo, Norway) according to the manufacturer's instructions. Isolated cells were washed three times in cold PBS and then subjected to RNA isolation.

RNA Blot Analysis-Total RNA was prepared from various cell lines, human PBMC's, PMN's, monocytes, PBL's, and day 1 and day 7 monocyte-derived Mφs using the acid guanidinium thiocyanate-phenol-chloroform method (Chomczynski and Sacchi, 1987). Total RNA (10 μg) was electrophoresed on a 1% formaldehyde-agarose gel, blotted to a nylon membrane (Duralon-UV, Stratagene) and hybridised with gene-specific probes as previously described (Lin et al., 1997). Likewise, commercially available human multiple tissue Northern blots (CLONTECH) were hybridised with gene-specific cDNA probes according to the manufacturer's instructions. Hybridised blots were washed in 0.5× SSC (0.15 M NaCl and 0.015 M sodium citrate), 1% SDS at 55 C. for 30 min and exposed to x-ray film (X-OMAT, Kodak) at −80 C. for 2-4 days. Radioactive probes were stripped from RNA blots by washing in 0.1× SSC/1% SDS for 15 min at 95 C. and the blots were rehybridised with a human β-actin cDNA probe to compare the amount of RNA loaded in each lane. Gene-specific cDNA probes used in the analysis include a 1.2 kb EMR2 3′-DNA fragment representing the 7TM region and a 230 bp CD97 cDNA fragment encoding the TM ¾ region.

RT-PCR-The expression profile of EMR2 and CD97 in various cell lines was evaluated by RT-PCR analysis using protocols described previously (Lin et al., 1997). EMR2-specific primers (5′-AGGTGCTCTGTGTCTTCTGGGA (SEQ ID NO. 11) and 5′-TTCTGGTTGGAGCCAGCGGGAAGGT (SEQ ID NO. 12)) as well as CD97-specific primers (5′-CGCACCACAAGAAAGTAGAGCTCCAG (SEQ ID NO. 13) and 5′-GGACTGGAAGCTGGACCCTGATCACCA (SEQ ID NO. 14)) were used to amplify EMR2 (600 bp) and CD97 (300 bp) cDNA fragments, respectively. Primers for β₂ microglobulin were used to amplify β₂ microglobulin cDNA as a positive and semi-quantitative control. In addition RT-PCR analysis was also conducted using RNA isolated from human peripheral blood leukocytes to identify alternatively spliced EMR2 transcripts. Primers used in the analysis include 5′-ACGGGATCCTCCTCCTGCACATCGTGGGCCATGT (SEQ ID NO.15) and 5′-AAACCATGGGAGGCCGCGTCTTTCTCGTCTTTCTCGCAATTCTGTGTCTGGC (SEQ ID NO. 16) for the extracellular domain, and 5′-AGGTGCTCTGTGTCTTCTGGGA (SEQ ID NO. 17) and 5′-TTCCACCGGCAAAGAGGGAAGATCTTATTC (SEQ ID NO.18) for the 7TM domain. PCR products were analysed on 1% agarose gels, purifid, subcloned and sequenced.

Construction of Expression Vectors-The EMR2 expression vectors containing 5 EGF-domains, EMR2 (EGF 1, 2, 3, 4, 5), and 3 EGF-domain, EMR2 (EGF 1, 2, 5))were constructed on the pcDNA3.1(+) (InVitrogen) backbone through the NotI-XbaI sites and HindIII-XbaI sites respectively. As a result of alternative splicing, the 5′end sequence containing 5 EGF-domains (EGF 1, 2, 3, 4, 5) or 3 EGF-domains (EGF 1, 2, 5) in conjunction with the stalk region was generated by RT-PCR as described above. The fragment containing 5 EGF domains was excised from the pCR2.1 vector with NotI and BamHI, and ligated with the EMR2 cDNA clone 5E/IC (isolated from the OriGene library) through the NotI-BamHI sites. The resulting full-length EMR2 ORF was excised by NotI and XbaI, and cloned into pcDNA3.1(+) through the NotI-XbaI sites to generate EMR2 (EGF 1, 2, 3, 4, 5). The EMR2 (EGF 1, 2, 5) expression vector was constructed by placing the HindIII-BamHI fragment of EMR2 (EGF 1, 2, 3, 4, 5) with the HindIII-BamHI pre-digested fragment containing EGF domains 1, 2 and 5. Both EMR2 (EGF 1, 2, 3, 4, 5) and EMR2 (EGF 1, 2, 5) were subjected to DNA sequencing of both strands to confirm their integrity. Hum CD97 expression constructs CD97 (EGF 1, 2, 3, 4, 5) and CD97 (EGF 1, 2, 5) were described elsewhere (Hamann et al., 1998).

Transient Transfection of Cells and Protein Analysis-Eukaryotic expression constructs encoding alternatively-spliced forms of human EMR2 and CD97 proteins [EMR2 (EGF 1, 2, 3, 4, 5), EMR2 (EGF 1, 2, 5), CD97 (EGF 1, 2, 3, 4, 5) and CD97 (EGF 1, 2, 5)] were transfected into CHO-K1 and COS-7 cells using Lipofectamine (Life Technologies, Inc.) according to the manufacturer's protocol. For FACS analysis, cells were harvested 48-72 hr post-transfection, fixed with 4% paraformaldehyde in PBS contaiing 100 mM HEPES (pH 7.3) for 30 min at 4° C., then quenched with culture medium containing 10% FCS at room temperature for 15 min. Cells were washed twice with PBS, blocked with 10% normal goat serum in PBS (blocking buffer) for 1 hr at 4° C., and reacted for 1 hr at 4° C. with mAbs (5 μg/ml) diluted in blocking buffer. Cells were then washed in PBS three times, and incubated with fluorescein isothiocyanate (FITC)-conjugated goat anti-mouse IgG (Jackson ImmunoResearch Laboratories, Inc.) diluted 1:100 in blocking buffer at 4° C. for 1 hr. After thee extensive washes in PBS, cells were resuspended in 200 μl and analysed on a FACScan. Data were collected and analysed using CellQuest software. For immunocytochemical analysis, transfected cells were harvested 24 hr post-transfection and plated on 11 mm diameter glass cover slips in 24 well plates at a density of 1×10⁵ cells/well. Cells were cultured for a further 48 hr before fixation. Immunostaining was then performed using the same protocols for FACS analysis as described above. The cover slips were mounted onto glass slides after the last wash using Vectashield mountant (Vector Laboratories), cells examined by fluorescence microscopy and representative photographs taken. For cell binding assays, transfected COS-7 cells were harvested 24 hr post-transfection, replated at 2×10⁵ cells/well in 6-well plates, and cultured for a further 2 days. Cells were washed three times with PBS, overlaid with 1×10⁸ human red blood cells (RBC) suspended in 1 ml serum free DMEM and incubated at room temperature for 30 min. Non-binding cells were gently removed by rinsing with PBS three times prior to examination by microscopy.

Results

In a search for new members of the EGF-TM7 family, BLAST programs were used to perform homology searches in the non-redundant DNA sequence database and expressed sequence tag (EST) databases. Among several candidate clones identified, two overlapping human cosmids, R29368 and F21185 (GenBank accession numbers AC004262 and AC005327), derived from human chromosome 19p13.1 locus, were identified. The coding sequence of the cosmids, predicted by the Grail program (Uberbacher and Mural, 1991; Uberbacher et al., 1996; Xu et al., 1994), indicates a putative protein sharing approximately 50% amino acid sequence identity with the 7TM regions of EMR1 and CD97 (data not shown). This information suggested the presence of a putative novel EGF-TM7 gene located in the vicinity of the EMR1 and CD97 loci on human chromosome 19p13. To clone the full-length cDNA, 5′- and 3′-RACE reactions as well as cDNA library screening were performed. As a result, a number of overlapping cDNA clones were identified. The complete cDNA sequence matches exactly the coding sequence within the cosmid clones and the deduced amino acid sequence predicted a novel member of the EGF-TM7 family. This gene was therefore designated EMR2 following the nomenclature of EMR1, the founding member of the EGF-TM7 family (Baud et al., 1995).

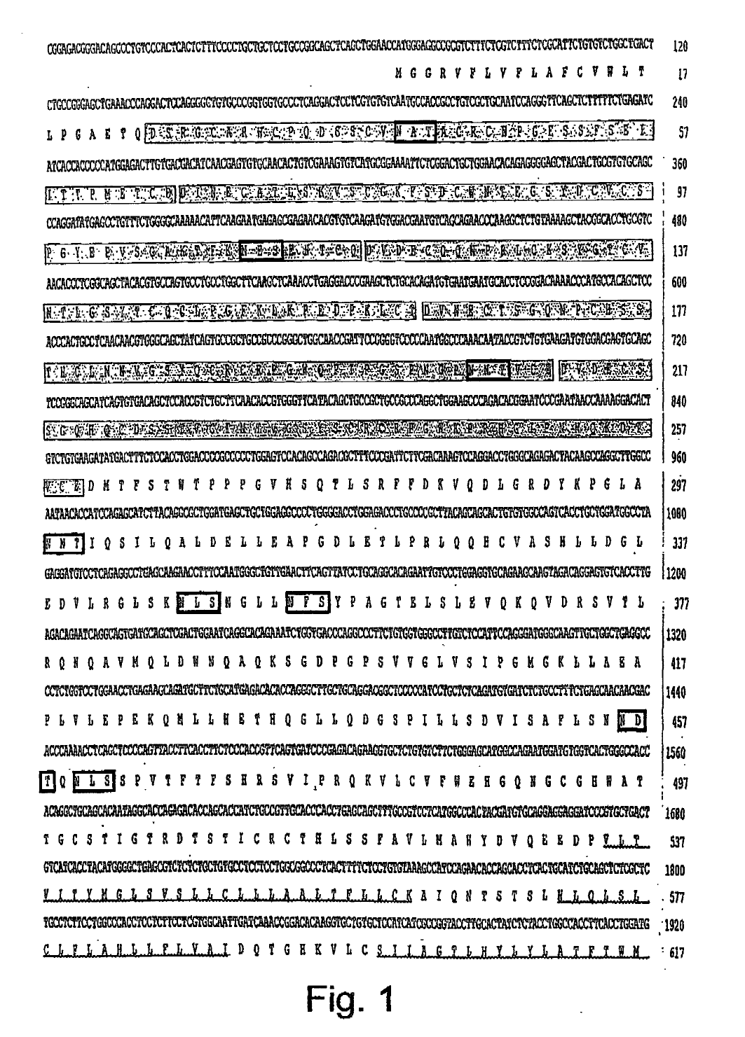

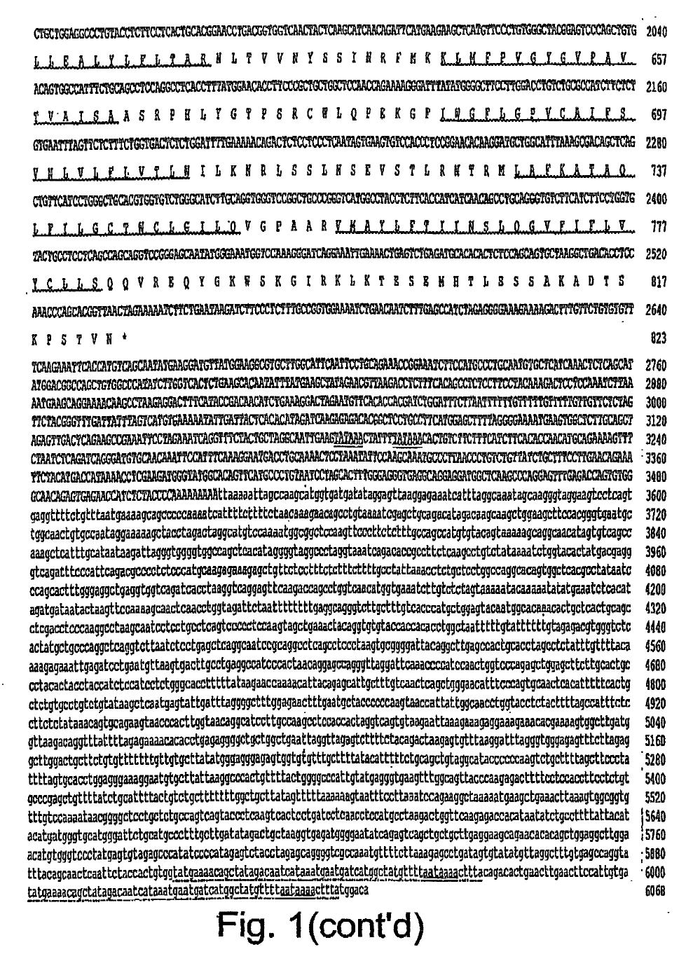

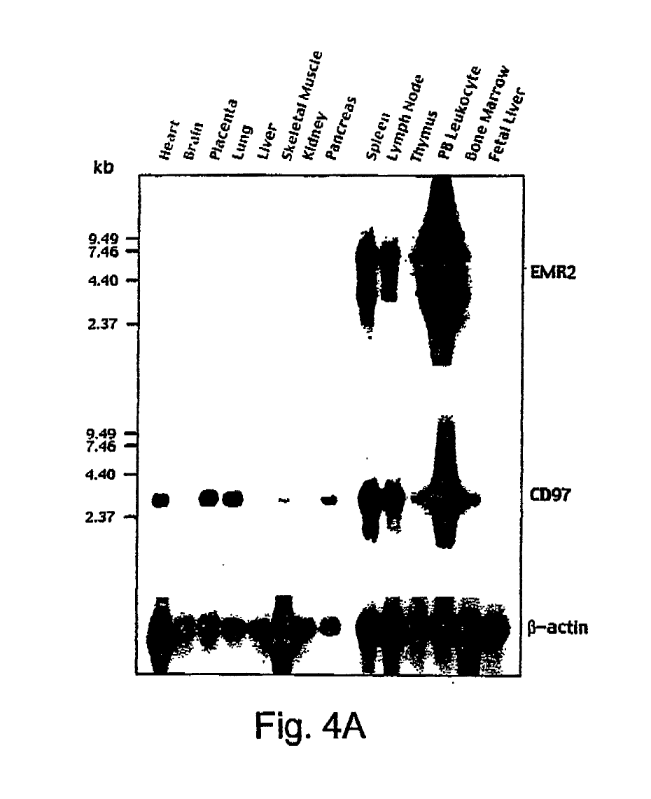

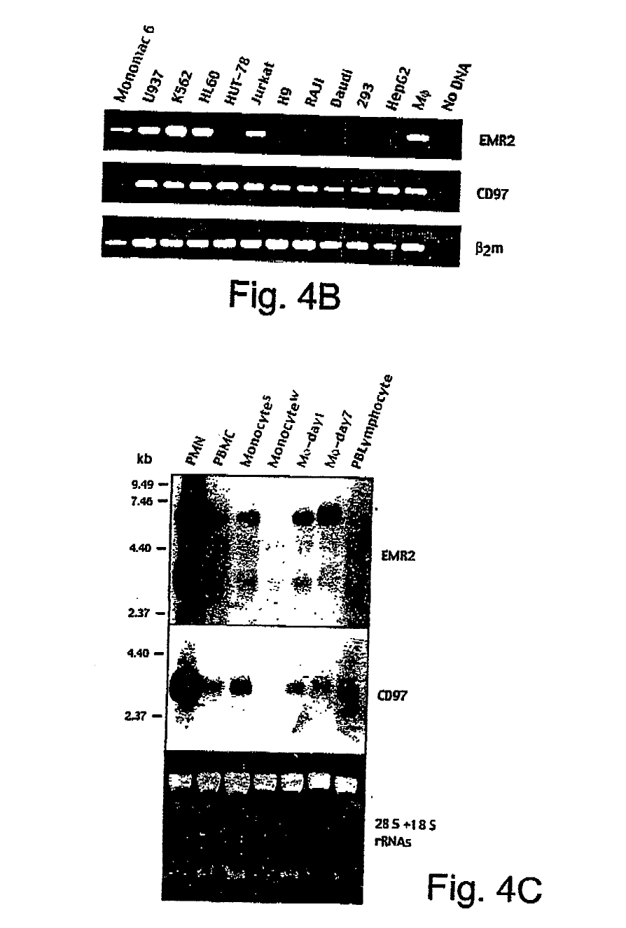

DNA sequence data and Northern blot analysis indicated two major EMR2 mRNA species, including a shorter transcript of 3.3 kb (SEQ ID NO. 19) and a longer transcript of 6.1 kb (SEQ ID NO. 1) (FIGS. 1 and 4). Both cDNA sequences share the same open reading frame (ORF) but differ in the length of their 3′-untranslated regions (UTR). The first in-frame ATG codon lies at position 70-72 and is preceded by an adenosine at position 67, which constitutes a favourable Kozak translation initiation motif (Kozak, 1987). An in-frame stop codon was identified at nt. 2539-2541. EMR2 cDNA therefore comprises a 5′UTR of 69 bp, a coding sequence of 2,469 bp and a 3′UTR of either 985 or 3530 bp (FIG. 1).

FIG. 1 shows the full-length cDNA sequence and deduced amino acid sequence of human EMR2. The open reading frame starts at nucleotide 70 and ends at nucleotides 2539-2541. The signal peptide sequence is highlighted in boldface lettering, the EGF domains are shaded and the seven transmembrane segments are underlined by crinkled lines. The potential N-glycosylation sites are boxed and the polyadenylation sites are double underlined. The DNA sequence represented by upper-case letters indicates both small and large transcripts with the additional 3′-UTR sequence of the large transcript represented by lower-case letters. Two perfect tandem repeats of 62 base pairs are underlined by dashed lines.

The differences in the length of the 3′UTR are thought to result from the differential usage of the polyadenylation signals. In addition, two perfect tandem repeats of 62 bp DNA sequence encompassing the polyadenylation sites are also contained within the longer transcript, though the significance of this feature is currently unknown.

A Kyte-Doolittle hydropathy profile of the predicted polypeptide sequence of 823 amino acids revealed a hydrophobic signal peptide at the N-terminus and seven hydrohobic segments at the C-terminus (data not shown). A predicted cleavage site at residue Thr²³ of the precursor protein implies that the resulting mature EMR2 protein is a cell surface molecule containing a long N-terminal extracellular region of 511 amino acids, a 7TM region of 248 amino acids and a cytoplasmic tail of 41 amino acids (FIG. 1).

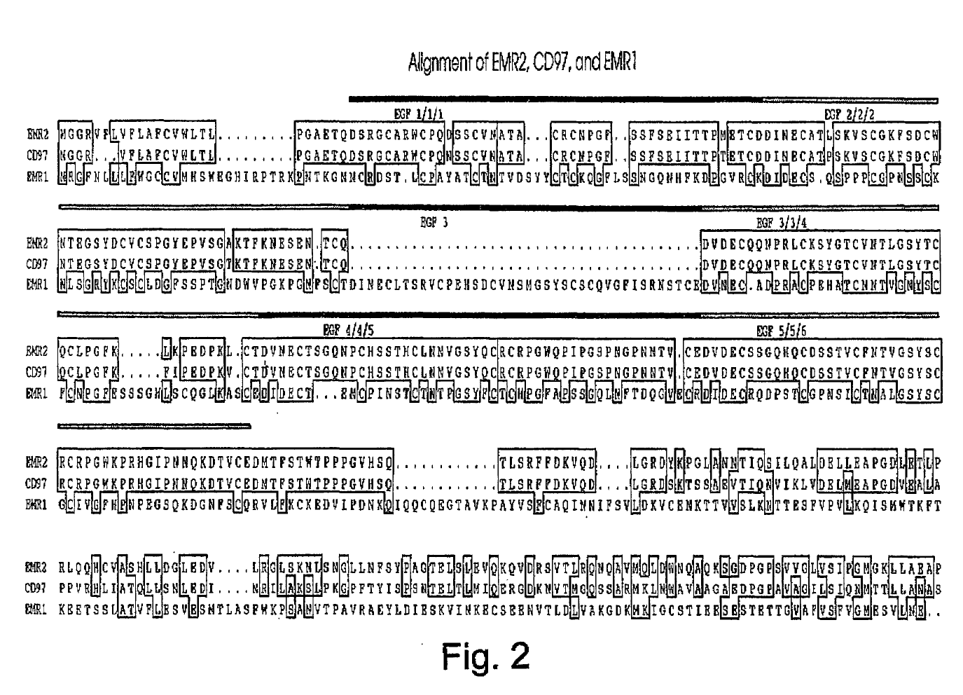

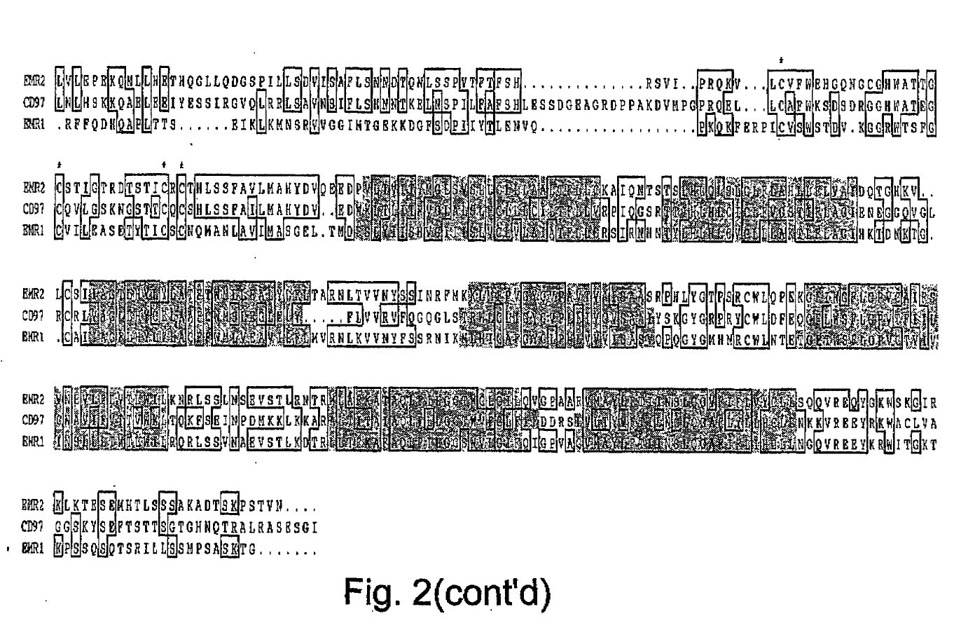

Amino acid sequence analysis reveals that EMR2 contains five EGF-like domains and is most closely related to CD97 (FIGS. 1 and 2) (Gray et al., 1996; Hamann et al., 1995). Surprisingly, the EGF domains of EMR2 are almost identical to those of CD97 with the sequence identity ranging from 95% to 100% in corresponding EGF domains. Of 236 amino acid residues in the five EGF domains of EMR2 and CD97, only two residues in domain 1, one residue in domain 2 and three residues in domain 3 are different. What is particularly surprising is that EMR2 only binds to CD55 with at least an order of magnitude less avidity than CD97, despite the similarity in sequence.

The high degree of identity in the EGF domain conserves the consensus amino acid sequences for the EGF domain calcium-binding site (DX[D/N]ECXnCXnCX[D/N]Xn[F/Y]XC) and for post-translational β-hydroxylation of aspartate/asparagine (CX[D/N]X₄[F/Y]XCXC), which were identified in EGF domains 2-5 (Gray et al., 1996; Hamann et al., 1995). Such calcium-binding EGF domains, found in a broad spectrum of extracellular proteins such as fibrillin-1 and -2, fibulin-1 and -2, thrombomodulin, protein-C and -S, factor-IX and -X and Drosophila proteins Notch, Delta and Serrate, have been shown to play important roles in protein-protein interactions involving cell adhesion, blood coagulation and receptor-ligand binding (Downing et al., 1996). Moreover, it has been previously demonstrated that the Ca²⁺-binding EGF domains are required for the interaction of CD97 and its cellular ligand, CD55 (Hamann et al., 1998). EMR2 therefore, like CD97, may interact with its ligand via the Ca²⁺ binding EGF domains.

Significant amino acid sequence homology between EMR2 and CD97 also extends to the spacer region (46% identity) and the 7TM region (45% identity). Multiple potential N- and O-glycosylation sites within the extracellular domain are found to be conserved as well (FIG. 1). Within the spacer region, a cysteine-rich motif of approximate 55 amino acids located immediately before the first TM segment was also recognised (FIG. 2).

FIG. 2 shows the amino acid sequence alignment of EMR2, CD97 and EMR1. The amino acid sequences of human EMR2, CD97 and EMR1 proteins are aligned for maximal homology. Dark and grey solid bars over the sequences indicate the appropriately numbered EGF domains. Transmembrane regions are shaded and identical amino acid residues are boxed. Asterisks indicate the cysteine and tyrosine residues conserved within the extracellular cysteine-rich domain proximal to the first transmembrane region.

This motif is characterised by four invariant cysteine residues and two conserved tryptophan residues and is found in other members of the EGF-TM7 family and family B GPCR-related proteins such as the (α-latrotoxin rector/CIRL members, CL1, CL2 and CL3 (Krasnoperov et al., 1997; Sugita et al., 1998). The cysteine-rich motif, named GPS for GPCR proteolytic site, is believed to be involved in the proteolytic cleavage of the CIRL receptor molecules. Interestingly, a similar post-translational modification has been identified for CD97, which is processed intracellularly during protein transfer to the cell surface (Gray et al., 1996). The presence of the cysteine-rich motif in EMR2 suggests that EMR2 may also be proteolytically cleaved in this way.

Other identified structural features include cysteine residues within extracellular loops 1 and 2 that are highly conserved among GPCR's and four consensus sequences for protein kinase C-mediated phosphorylation in intracellular loops 2 and 3 and the cytoplasmic tail (TAR at aa 627-629, TLR at aa 723-725, SAK at aa 811-813 and TSK at aa 816-818).

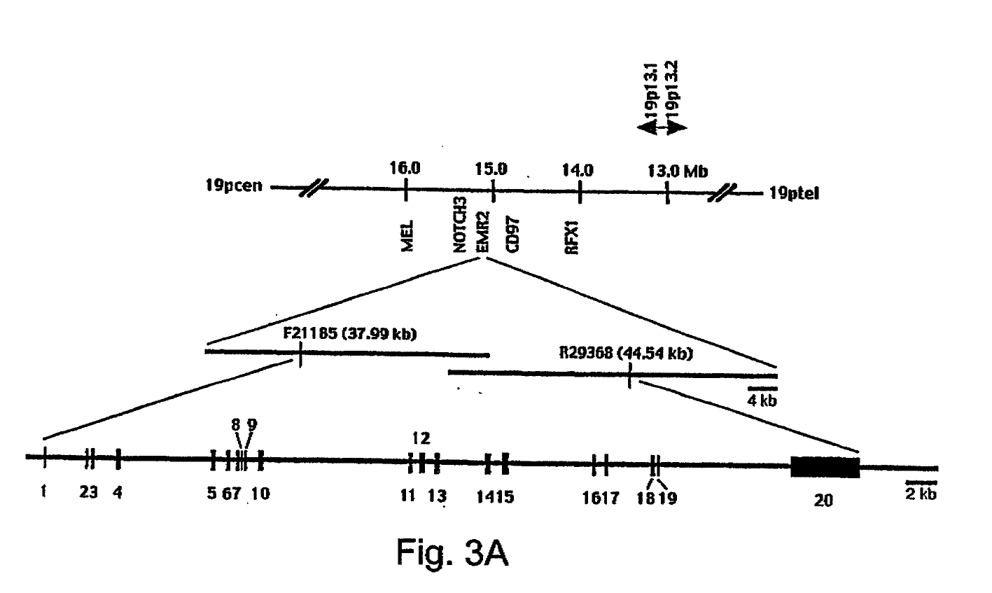

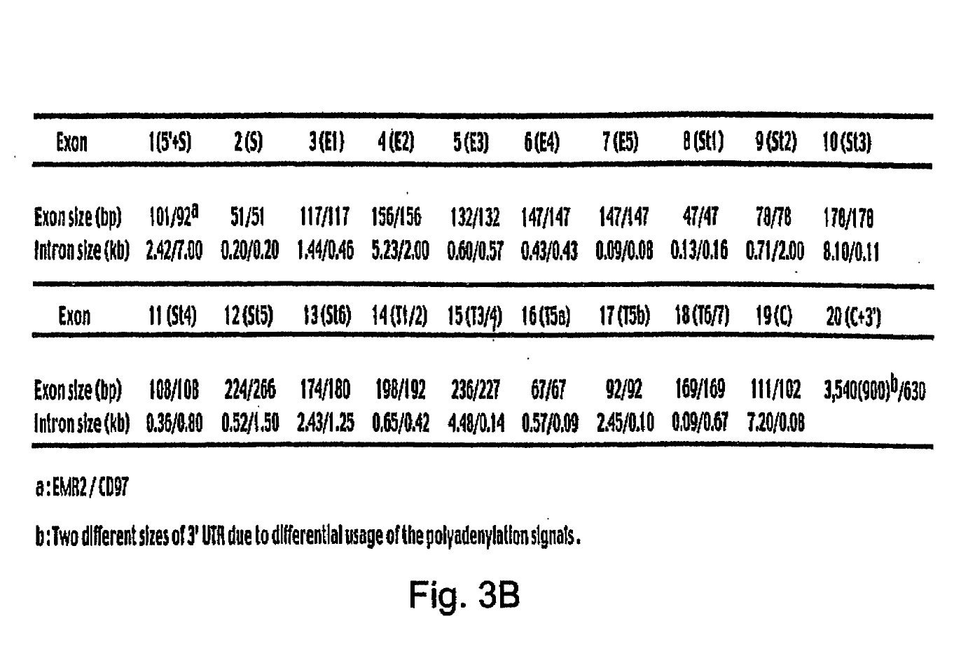

The entire EMR2 gene, containing 20 exons, is approximately 45 kb in length and is localised between the NOTCH3 and RFX1 loci on chromosome 19p13.1, a region to which CD97 has previously been mapped (FIG. 3) (Carver et al., 1999).

FIG. 3 shows the location and structure of the EMR2 gene on human chromosome 19p13.1. A) The location of EMR2 gene is positioned according to the latest chromosome mapping information (http://www-bio.llnl.gov/bbrp/genome/genome.html.). Scales above the line indicate the distance from the p-telomere in Mb. Cosmids F21185 and R29368 are represented by solid bars and the exons are indicated by numbered vertical bars. B) Comparison of the exon-intron organisation of the human EMR2 and CD97 genes. The sizes of the exons and introns comprising the EMR2 and CD97 genes are compared in base pairs and kilobase pairs, respectively. The abbreviations used 5′, 5′-untranslated region; S, the signal peptide; E, EGF domain; St; the stalk region; T, transmembrane segment; C, cytoplasmic tail; 3′, 3′-untranslated region.

The exon-intron organisation of the EMR2 and CD97 genes was found to be identical with each EGF domain encoded by a single exon and the 7TM domain encoded by a total of five exons. Intriguingly, the majority of the corresponding exons of EMR2 and CD97 are of the same size; of the 20 exons identified, only exons 12, 13, 14, 15 and 19 are of slightly different sizes (FIG. 3B). These results demonstrate a highly conserved gene organisation between EMR2 and CD97 and strongly suggest that the EMR2 nd CD97 genes were derived from a recent gene duplication event.

Previously described members of the EGF-TM7 family, EMR1, F4/80 and CD97, have all been shown to be highly expressed by leukocytes, with abundant levels on myeloid cells (McKnight and Gordon, 1996). Northern blot analysis of multiple human tissues demonstrated that EMR2 is expressed most abundantly in peripheral blood leukocytes followed by high levels of expression in spleen and lymph nodes, and intermediate to low levels of expression in thymus, bone marrow, foetal liver, placenta and lung (FIG. 4A).

FIG. 4 shows the tissue and cell-type expression patterns of EMR2. A) Northern blots containing equal amounts of poly(A)⁻-RNA from the indicated human tissues were sequentially hybridised with ³²P-labelled probes specific for EMR2, CD97 and β-actin. Two EMR2 transcripts, resulting from differential polyadenylation, were detected. Molecular weight markers are indicated on the left. B) Messenger RNA isolated from a series of human cell lines and from day 7 human monocyte-derived macrophages was used as template for RT-PCR analysis, as described in Materials and Methods. A PCR reaction without DNA template was used as negative control. C) Total RNA (10 μg) from human PMN's, PBMC's, strongly and weakly adherent monocytes (Monocyte^{s} and Monocyte^{w}), peripheral blood lymphocytes (PBLymphocyte) as well as day 1 and day 7 monocyte-derived macrophages (Mφ-day 1 and Mφ-day 7) was analysed by Northern blotting as described in Materials and Methods. Numbers on the left indicate positions of molecular weight markers. Ethidium bromide staining of the gel in the bottom panel shows the equal loading and integrity of the RNA samples.

A very weak signal can also be identified in liver, however no signal was detected in heart, brain, skeletal muscle, kidney or pancreas. CD97 showed a similar expression pattern in immune tissues but also expressed high to intermediate levels in heart, skeletal muscle, kidney and pancreas. This result suggests that EMR2 expression may be restricted to cells of certain hematopoietic lineages.

RT-PCR analysis was then carried out using various hamatopoietic cell lines to investigate the restricted expression pattern of EMR2 (FIG. 4B). Among 11 cell lines examined EMR2 expression was detected in all monocyte/Mφ cell lines and one T cell line (MonoMac 6, U937, K562, HL60 and Jurkat) but not in the other cell lies tested (HUT-78, H9, RAJI Daudi, HEK293 and HepG2 cells). In contrast, CD97 expression was detected in all of the cell lines tested, confirming that EMR2 expression is restricted. Next, we set out to identify the EMR2-expressing blood cell types within the peripheral blood leukocyte population as a first step towards understanding the function of EMR2. Northern blot analysis showed that EMR2 is very strongly expressed in PMN's and at lower levels in freshly isolated strongly-adherent monocytes (FIG. 4C). Furthermore, EMR2 expression in monocytes is up-regulated following maturation in culture and reached a higher expression level at day 7. Interestingly, the shorter EMR2 transcript is down-regulated during monocyte differentiation in culture, suggesting the possibility of different regulatory mechanisms governing the expression/stability of the short and long EMR2 transcripts.

EMR2 transcripts were barely detectable in PBMC's, weakly-adherent monocytes or peripheral blood lymphocytes; the hybridisation signal detected in these cells is probably due to low levels of contaminating monocytes. CD97 showed a similar expression pattern to EMR2 in PMN's, monocytes and Mφ, however, CD97 expression could also be detected in PBMC's, weakly-adherent monocytes and peripheral blood lymphocytes. These expression studies establish that EMR2 is restricted to granulocytic and monocyte/Mφ lineages, whereas CD97 is ubiquitously expressed in all blood cell types examined. In addition, it indicated that EMR2 expression is highly regulated during monocyte/Mφ differentiation.

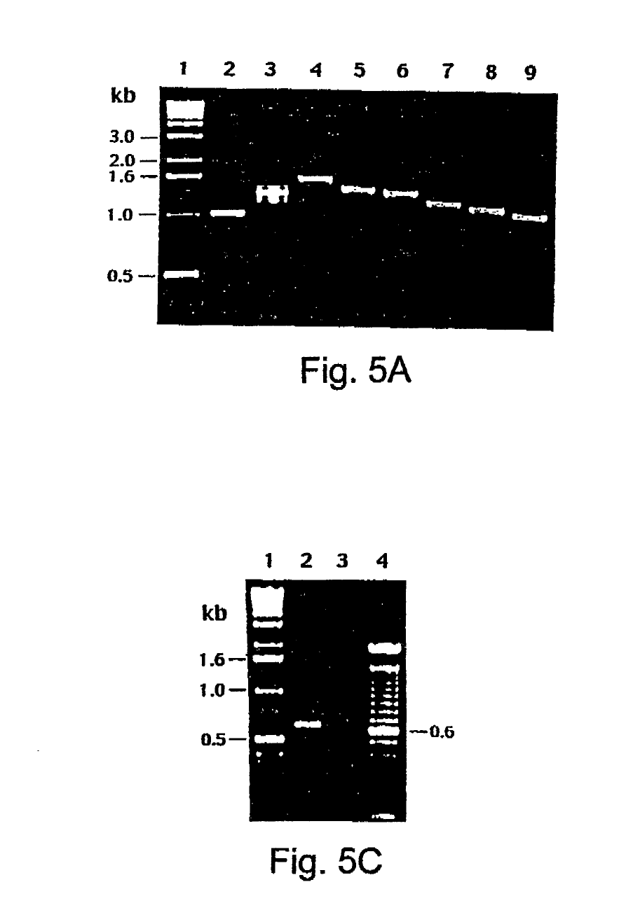

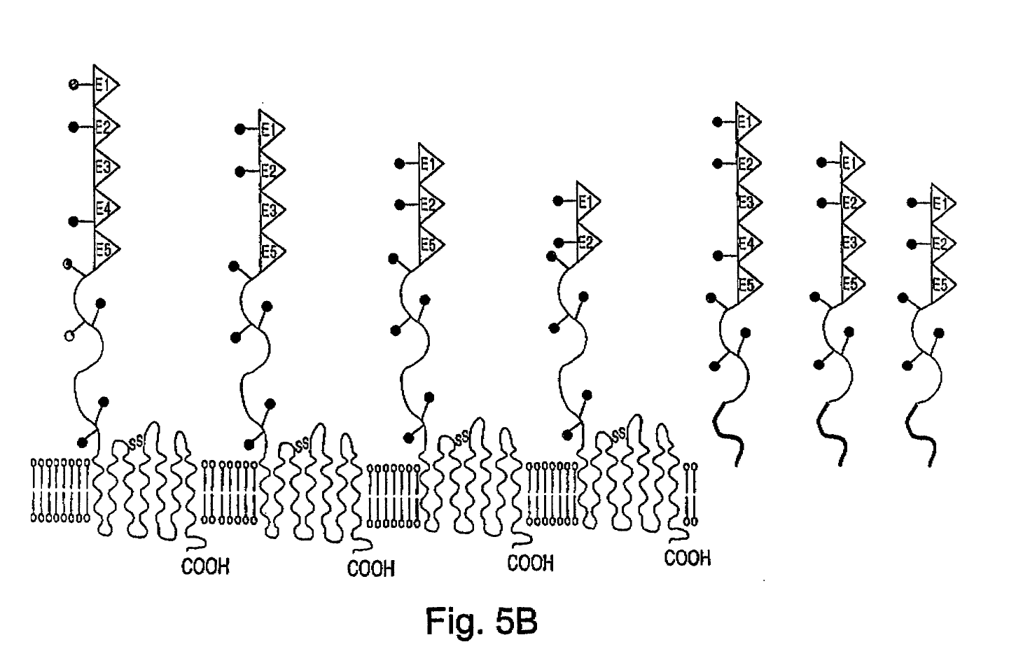

Another characteristic of EGF-TM7 genes is the presence of extensive alternatively spliced mRNA transcripts. Alternative splicing has been found to occur predominantly at the 5′ end of the transcripts, potentially resulting in multiple protein isoforms that contain different numbers and/or combinations of EGF-like domains (Gray et al., 1996; Lin et al., 1997; McKnight and Gordon, 1998). RT-PCR analysis and sequence analysis indeed revealed multiple alternatively spliced forms of EMR2 mRNA (FIG. 5).

FIG. 5 shows alternative splicing of EMR2 RNA transcripts. A) Lane 2 shows the RT-PCR product generated using primers specific for the DNA encoding the seven-transmembrane region. Lane 3 shows the multiple DNA fragments generated using primers specific for the extracellular domain. Lanes 4-9 indicate the individual DNA fragments isolated from lane 3. Numbers on the left indicate the molecular weight markers used in lane 1. B) Schematic structures of the EMR2 protein isoforms resulting from alternative RNA splicing. Four putative transmembrane protein isoforms and three soluble protein isoforms are predicted. The EGF-like domains are represented by a capital letter E, followed by a number. The bold line indicates the frame-shifted amino acid sequence of the putative soluble protein isoforms. C) RT-PCR analysis conforms the alternatively spliced transcripts encoding the soluble EMR2 protein isoforms. Lane 2 shows the specific PCR product of approximately 0.65 kb. Lane 3 is the negative control with no DNA template. Numbers shown indicate the molecular weight markers used in lanes 1 and 4.

Similar to CD97, putative EMR2 protein isoforms containing 5 EGF domains (EGF 1, 2, 3, 4, 5), 4 EGF domains (EGF 1, 2, 3, 5), 3 EGF domains (EGF 1, 2, 5) and 2 EGF domains (EGF 1, 2) are predicted.

Unexpectedly, however, a splice variant resulting from the bypass of exon 12 was also identified and predicted to encode a soluble EMR2 molecule due to a frameshift, which introduced a stop codon immediately before the fist TM domain (FIG. 5C).

In addition, another alternative splicing event resulting from the utilization of different splicing acceptor sites in exon 12 was also identified. As a result, an EMR2 transmembrane molecule with an 11 amino acid deletion in the spacer domain is predicted (data not shown). Such a deletion mutant is another preferred feature of the invention. Similar alternative splicing of exons encoding the spacer and transmembrane regions of CD97 has also been observed. To date we have identified at least 7 alternative EMR2 transcripts by RT-PCR in peripheral blood leukocytes (FIG. 5B).

EMR2 may be detected by mAbs recognising the EGF-like domains of CD97 but not by mAbs specific for the spacer region of CD97. This was verified by transfecting cells with either EMR2 or CD97 expression constructs and subjecting them to FACS analysis and immunocytochemistry using the following mAbs; BL-Ac/F2, CLB-CD97/1, CLB-CD97/2 and CLB-CD97/3. Both BL-Ac/F2 and CLB-CD97/1 had previously been shown to recognise the EGF-like domain 1 of human CD97, whereas CLB-CD97/2 and CLB-CD97/3 were found to be specific for the spacer region of CD97 (Hamann et al., 1998).

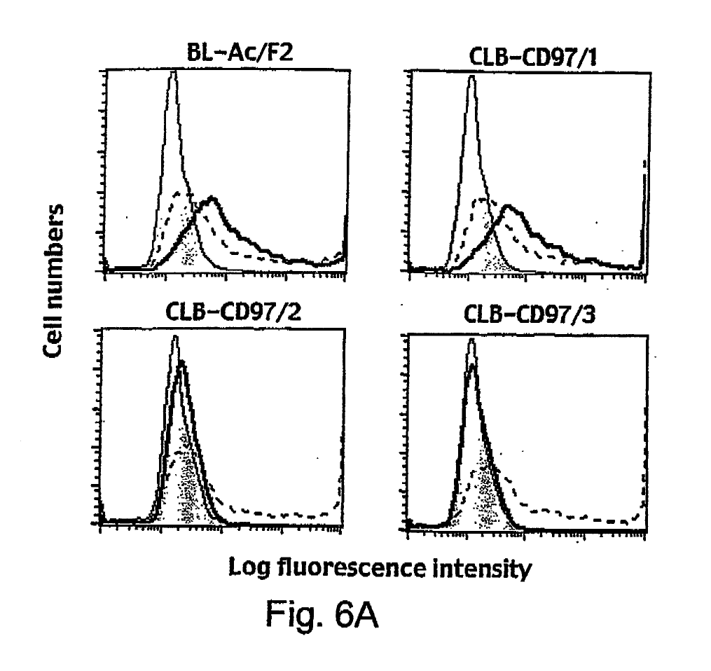

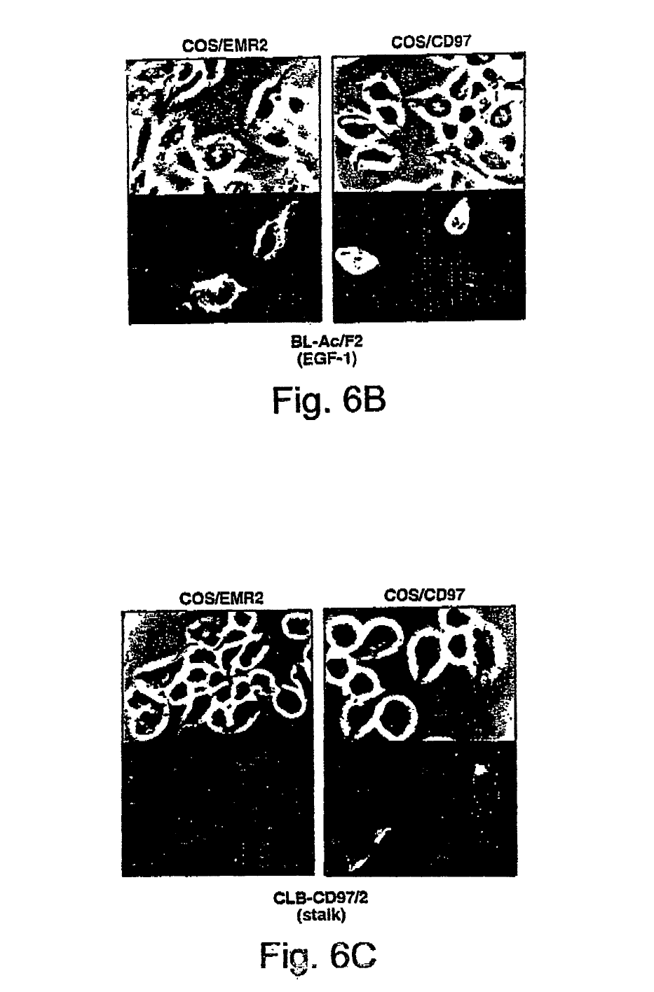

When stained with BL-Ac/F2 and CLB-CD97/1, a similar percentage of EMR2-transfected cells exhibited comparable levels of cell surface reactivity as the cells transfected with the CD97 expression construct (FIG. 6).

FIG. 6 shows cell surface expression of EMR2 on transfected cells. A) Flow cytometric analysis of CHO-K1 cells expressing either EMR2 (solid line) or CD97 (dash line) expression constructs was performed with the mAbs BL-Ac/F2, CLB-CD97/1, CLB-CD97/2 and CLB-CD97/3. Mock-transfected CHO-K1 cells were used as the negative control (filled space). B and C) lmmunocytochemical analysis of COS-7 cells expressing either EMR2 (COS/EMR2) or CD97 (COS/CD97) expression constructs was performed with the mAbs BL-Ac/F2 and CLB-CD97/2. The phase contrast and the corresponding fluorescence photographs are shown at the upper and lower panels respectively. The mAbs CLB-CD97/1 and CLB-CD97/3 showed a similar staining pattern as did BL-Ac/F2 and CLB-CD97/2 (data not shown).

In contrast, mAbs CLB-CD97/2 and CLB-CD97/3 only reacted with CD97-transfected cells and not at all with EMR2-transfected cells (FIG. 6). Thus, both EMR12 and CD97 can be recognised by mAbs specific for the highly homologous EGF-like domain but they can also be distinguished by the epitopes within the extracellular spacer domain.

The highly homologous amino acid sequences of the EGF-like domains of EMR2 and CD97 suggest that EMR2 would interact with CD55. It had been shown previously that at least three tandemly linked EGF-like domains are required for CD97-CD55 binding and that the shortest CD97 isoform CD97 (EGF 1, 2, 5) has a significantly higher affinity for CD55 than the larger isoforms CD97 (EGF 1, 2, 3, 5) and CD97 (EGF 1, 2, 3, 4, 5) (Hamann et al., 1998). Two EMR2 expression constructs, EMR2 (EGF 1, 2, 5) and EMR2 (EGF 1, 2, 3, 4, 5), were therefore used in conjunction with CD97 (EGF 1, 2, 5) and CD97 (EFG 1, 2, 3, 4, 5) in a cell rosetting assay to determine whether EMR2 could bind to CD55. The expression of both EMR2 and CD97 isoforms at the cell surface of transfected cells was confirmed by FACS analysis and shown to be at similar levels (data not shown).

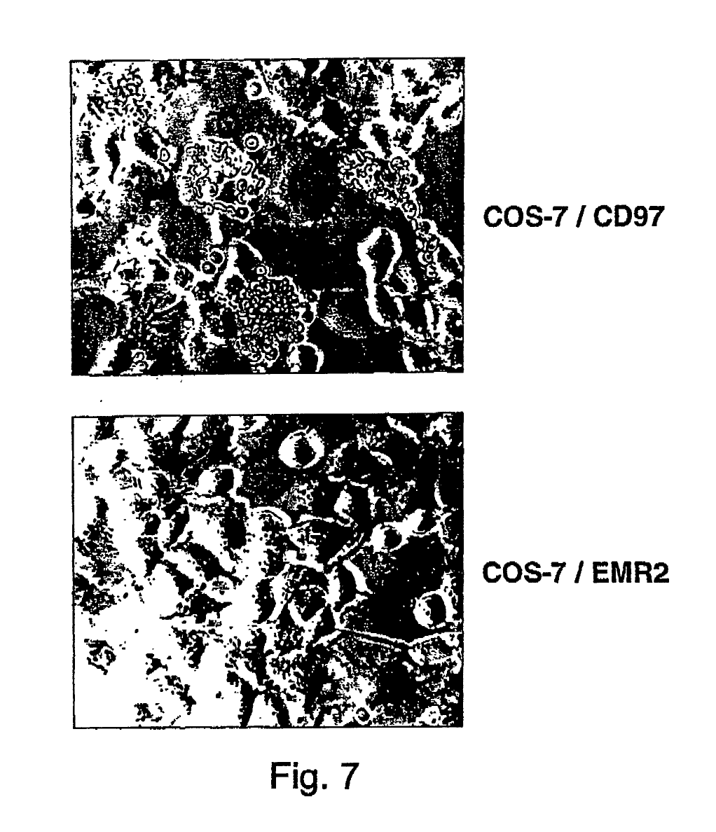

FIG. 7 shows that EMR2 does not interact with CD55. Human red blood cells were overlaid on CD97transfected (COS-7/CD97) or EMR2-transfected (COS-7/EMR2) COS-7 cells and incubated for 30 minutes prior to three extensive washes. Neither EMR2 (EGF 1, 2, 5)- nor EMR2 (EGF 1, 2, 3, 4, 5)-transfected cells rosetted human red blood cells. This experiment was repeated three times with the same result.

As expected, CD97 (EGF 1, 2, 5) transfected cells (FIG. 7) bound to human red blood cells (RBC), a rich source of CD55 ligand, much more efficiently than did the CD97 (EGF 1, 2, 3, 4, 5)-transfected cells (data not shown). Surprisingly, however, neither EMR2 (EGF 1, 2, 5) nor EMR2 (EGF 1, 2, 3, 4, 5)-transfected cells bound human RBC (FIG. 7). Since the EGF-like domains of the EMR2 (EGF 1, 2, 5) and CD97 (EGF 1, 2, 5) differ by only three amino acid residues, this intriguing result implies that the CD97-CD55 interaction is highly specific and that the binding specificity is dependent upon these three (or fewer) amino acids.

Abe, J., H. Suzuki, M. Notoya, T. Yamamoto, and S. Hirose. (1999). Ig-hepta, a novel member of the G protein-coupled hepta-helical receptor (GPCR) family that has immunoglobulin-like repeats in a long N-terminal extracellular domain and defines a new subfamily of GPCRS. J Biol. Chem. 274:19957-19964.

Baud, V., S. L. Chissoe, B. Viegas-Pequignot, S. Diriong, V. C. N'Guyen, B. A. Roe, and M. Lipinski. (1995). EMR1, an unusual member in the family of hormone receptors with seven transmembrane segments. Genomics. 26:334-344.

Bockaert, J., and J. P. Pin. (1999). Molecular tinkering of G protein-coupled receptors: an evolutionary success. EMBO J. 18;1723-1729.

Boulay, F., M. Tardif, L. Brouchon, and P. Vignais. (1990). The human N-formylpeptide receptor. Characterisation of two cDNA isolates and evidence for a new subfamily of G-protein-coupled receptors. Biochemistry. 29:11123-11133.

Carver, E. A., J. Hamann, A. S. Olsen, and L. Stubbs. (1999). Physical mapping of EMR1 and CD97 in human chromosome 19 and assignment of Cd97 to mouse chromosome 8 suggest an ancient genomic duplication [In Process Citation]. Mamm Genome. 10:1039-1040.

Chomczynski, P., and N. Sacchi. (1987). Single-step method of RNA isolation by acid guanidinium thiocyanate- phenol-chloroform extraction Anal Biochem. 162:156-159.

Downing, A. K., V. Knott, J. M. Werner, C. M. Cardy, I. D. Campbell, and P. A. Handford. (1996). Solution structure of a pair of calcium-binding epidermal growth factor-like domains: implications for the Marfan syndrome and other genetic disorders. Cell. 85:597-605.

Eichler, W., G. Aust, and D. Hamann. (1994). Characterisation of an early activation-dependent antigen on lymphocytes defined by the monoclonal antibody BL-Ac(F2). Scand J Immunol. 39:111-115.

Eichler, W., J. Hamann, and G. Aust. (1997). Expression characteristics of the human CD97 antigen. Tissue Antigens. 50:429-438.

6++Gerard, C., and N. P. Gerard (1994). C5A anaphylatoxin and its seven transmembrane-segment receptor. Annu Rev Immunol. 12:775-808.

Gray, J. X., M. Haino, M. J. Roth, J. E. Maguire, P. N. Jensen, A. Yarme, M. A. Stetler-Stevenson, U. Slebenlist, and K. Kelly. (1996). CD97 is a processed, seven-transmembrane, heterodimeric receptor associated with inflammation. J Immunol. 157:5438-5447.

Hadjantonakis, A. K., C. J. Formstone, and P. F. R. Little. (1998). mCelsrl is an evolutionarily conserved seven-pass transmembrane receptor and is expressed during mouse embryonic development. Mech Dev. 78:91-95.

Hamann, J., W. Eichler, D. Hamann, H. M. Kerstens, P. J. Poddighe, J. M. Hoovers, E. Hartmann, M. Strauss, and R. A. van Lier. (1995). Expression cloning and chromosomal mapping of the leukocyte activation antigen CD97, a new seven-span transmembrane molecule of the secretion receptor superfamily with an unusual extracellular domain. J Immunol. 155:1942-1950.

Hamann J., E. Hartmann, and R. A. van Lier. (1996a). Structure of the human CD97 gene: exon shuffling has generated a new type of seven-span transmembrane molecule related to the secretin receptor superfamily. Genomics. 32:144-147.

Hamann, J., C. Stortelers, E. Kiss-Toth, B. Vogel, W. Eichler, and R. A. van Lier. (1998). Characterisation of the CD55 (DAF)-binding site on the seven-span transmembrane receptor

CD97. Eur J Immunol. 28:1701-1707.

Hamann, J., B. Vogel, G. M. van Schijndel, and R. A. van Lier. (1996b). The seven-span transmembrane receptor CD97 has a cellular ligand (CD55, DAF). J Exp Med. 184:1185-1189.

Herbein, G., A. G. Doyle, L. J. Montaner, and S. Gordon. (1995). Lipopolysaccharide (LPS) down-regulates CD4 expression in primary human macrophages through induction of endogenous tumour necrosis factor (TNF) and IL-1 beta. Clin Exp Immunol. 102:430-437.

Hoang-Vu, C., K. Bull, I. Schwarz, G. Krause, C. Schmutzler, G. Aust, J. Kohrle, and H. Dralle. (1999). Regulation of CD97 protein in thyroid carcinoma J Clin Endocrinol Metab. 84:1104-1109. Holmes, W. E., J. Lee, W. J. Kuang, G. C. Rice, and W. I. Wood. 1991. Structure and functional expression of a human interleukin-8 receptor. Science. 253:1278-1280.

Kozak M. (1987). An analysis of 5′-noncoding sequences from 699 vertebrate messenger RNAS. Nucleic Acids Res. 15:8125-8148.

Krasnoperov, V. G., M. A Bittner, R. Beavis Y. Kuang, K. V. Salnikow, O. G. Chepurny, A. R. Little, A. N. Plotnikov, D. Wu, R. W. Holz, and A. G. Petrenko. (1997). alpha-Latrotoxin stimulates exocytosis by the interaction with a neuronal G-protein-coupled receptor. Neuron. 18:925-937. Lelianova, V. G., B. A. Davletov, A. Sterling, M. A. Rahman, E. V. Grishin, N. F. Totty, and Y. A. Ushkaryov. (1997). Alpha-latrotoxin receptor, latrophilin, is a novel member of the secretin family of G protein-coupled receptors. J Biol Chem. 272:21504-21508.

Lin, H. H., L. J. Stubbs, and M. L. Mucenski. (1997). Identification and characterisation of a seven transmembrane hormone receptor using differential display. Genomics. 41:301-308.

Liszewski, M. K., T. C. Farries, D. M. Lublin, I. A. Rooney, and J. P. Atkinson. (1996). Control of the complement system. Adv Immunol. 61;201-283.

Liu, M., R. M. Parker, K. Darby, H. J. Eyre, N. G. Copeland, J. Crawford, D. J. Gilbert, G. R. Sutherland, N. A. Jenkins, and H. Herzog. (1999). GPR56, a novel secretin-like human G-protein-coupled receptor gene. Genomics.55:296-305.

McKnight, A. J., and S. Gordon. (1996). EGF-TM7: a novel subfamily of seven-transmembrane-region leukocyte cell-surface molecules. Immunol Today. 17:283-287.

McKnight, A. J., and S. Gordon. (1998). The EGF-TM7 family: unusual structures at the leukocyte surface. J Leukoc Biol. 63:271-280.

McKnight, A. J., A. J. Macfarlane, P. Dri, L. Turley, A. C. Willis, and S. Gordon. (1996). Molecular cloning of F4/80, a murine macrophage-restricted cell surface glycoprotein with homology to the G-protein-linked transmembrane 7 hormone receptor family. J Biol Chem. 271:486-489.

McKnight, A. J., A. J. Macfarlane, M. F. Seldin, and S. Gordon. (1997). Chromosome mapping of the Emrl gene. Mamm Genome. 8:946.

Murphy, P. M., and D. McDermott. (1991). Functional expression of the human formyl peptide receptor in Xenopus oocytes requires a complementary human factor. J Biol Chem. 266:12560-12567.

Murphy, P. M., and H. L. Tiffany. (1991). Cloning of complementary DNA encoding a functional human interleukin-8 receptor. Science. 253:1280-1283.

Nishimori, H., T. Shiratsuchi, T. Urano, Y. Kimura, K Kiyono, K. Tatsumi, S. Yoshida, M. Ono, M. Kuwano, Y. Nakamura, and T. Tokino. (1997). A novel brain-specific p53-target gene, BAI1, containing thrombospondin type 1 repeats inhibits experimental angiogenesis. Oncogene. 15:2145-2150.

Osterhoff, C., R. Ivell, and C. Kirchhoff. (1997). Cloning of a human epididymis-specific nRNA, HE6, encoding a novel member of the seven transmembrane-domain receptor superfamily. DNA Cell Biol. 16:379-389.

Qian, Y. M., M Haino, K. Kelly, and W. Song. (1999). Structural characterisation of mouse CD97 and study of its specific interaction with the murine decay-accelerating factor (DAF, CD55) [In Process Citation]. Immunology. 98:303-311.

Shiratsuchi, T., H. Nishimori, H. Ichise, Y. Nakamura, and T. Tokino. (1997). Cloning and characterisation of BAI2 add BAI3, novel genes homologous to brain-specific angiogenesis inhibitor 1 (BAI1). Cytogenet Cell Genet. 79:103-108.

Sugita, S., K. Ichtchenko, M. Khvotchev, and T. C. Sudhof. (1998). alpha-Latrotoxin receptor CIRL/latrophilin 1 (CL1) defines an unusual family of ubiquitous G-protein-linked receptors. G-protein coupling not required for triggering exocytosis. J Biol Chem. 273:32715-32724.

Uberbacher, E. C., and R. J. Mural. (1991). Locating protein-coding regions in human DNA sequences by a multiple sensor-neural network approach. Proc Natl Acad Sci USA 88:11261-11265.

Uberbacher, E. C., Y. Xu, and R. J. Mural. (1996). Discovering and understanding genes in human DNA sequence using GRAIL. Methods Enzymol. 266:259-281.

Wess, J. (1997). G-protein-coupled receptors: molecular mechanisms involved in receptor activation and selectivity of G-protein recognition. FASEB J. 11:346-354.

Xu, Y., R. Mural, M. Shah, and E. Uberbacher. (1994). Recognising exons in genomic sequence using GRAIL IL Genet Eng. 16:241-253.

Zendman A. J., I. M. Cornelissen, U. H. Weidle, D. J. Ruiter, and G. N. van Muijen. (1999). TM7XN1, a novel human EGF-TM7-like cDNA, detected with mRNA differential display using human melanoma cell lines with different metastatic potential. FEBS Lett. 446:292-298.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. The use of an EMR2 polypeptide-type polypeptide in the preparation of a medicament for use in a method of therapy of a condition or disease associated with an interaction between the EMR2 polypeptide and an EMR2 ligand, wherein the EMR2 polypeptide-type polypeptide comprises a domain corresponding to the EGF4 domain of EMR2, and which binds to an antibody specific to the EGF4 domain.

2. The use of an EMR2 polypeptide-type polypeptide in the preparation of a diagnostic agent for use in a method of diagnosis of a condition disease associated with an interaction between the EMR2 polypeptide and an EMR2 ligand, wherein the EMR2 polypeptide-type polypeptide comprises a domain corresponding to the EGF4 domain of EMR2, and which binds to an antibody specific to the EGF4 domain.

3. A use according to any preceding claim, wherein the EMR2 polypeptide-type polypeptide comprises all or part of the sequence of SEQ ID NO. 2, or a variant thereof.

4. A use according to any preceding claim, wherein the EMR2 polypeptide-type polypeptide comprises all or part of the sequence of SEQ ID NO. 20, or a variant thereof.

5. A use according to any preceding claim, wherein the EMR2 polypeptide-type polypeptide comprises up to 15 EGF subunits.

6. A use according to any preceding claim, wherein the EMR2 polypeptide-type polypeptide comprises up to 10 EGF subunits.

7. A use according to any preceding claim, wherein the EMR2 polypeptide-type polypeptide comprises 5 EGF subunits.

8. A use according to any preceding claim, wherein the EMR2 polypeptide-type polypeptide comprises at least one EGF subunit, and wherein at least one EGF subunit corresponds to EGF subunit 4 of naturally occurring EMR2, or a variant thereof.

9. A use according to claim 8, wherein two or more EGF subunits are tandem or spaced apart repeats of EGF4.

10. A use according to claim 8, wherein the EMR2 polypeptide-type polypeptide comprises 5 EGF subunits and there is only one EGF4 sequence, said EGF4 sequence being in the 4^{th} position of the 5 EGF subunits.

11. A use according to any preceding claim, wherein the EMR2 polypeptide-type polypeptide substantially lacks any transmembrane sequences.

12. A use according to any preceding claim, wherein the polypeptide-type polypeptide is soluble in saline.

13. A use according to any preceding claim, wherein the EMR2 polypeptide-type polypeptide is substantially identical with the sequence of SEQ ID NO. 2, where the sequences correspond.

14. A use according to any preceding claim, wherein the EMR2 polypeptide-type polypeptide is not glycosylated.

15. An agonist for the polypeptide of SEQ ID NO. 2 or 20.

16. An antagonist for the polypeptide of SEQ ID NO. 2 or 20.

17. An antibody specific for the polypeptide of SEQ ID NO. 2 or 20.

18. An antibody specific for the polypeptide of SEQ ID NO. 2 or 20 which is a monoclonal antibody.

19. An antibody according to claim 17 or 18 which is specific for the EGF 4 subunit of EMR2.

20. A polynucleotide, or a variant thereof, encoding an EMR2 polypeptide-type polypeptide which is
EMR2 polypeptide protein comprising the amino acid sequence of SEQ ID NO. 2 or SEQ ID NO. 20;
a polypeptide which has substantial identity therewith and which retains an EMR2 polypeptide biological activity; or
a fragment of such an EMR2 polypeptide protein or substantially identical polypeptide, and which fragment retains an EMR2 polypeptide biological activity.

21. An antisense polynucleotide to the polynucleotide of claim 20.

22. A polynucleotide according to claims 20 or 21, which is DNA.

23. A vector comprising a polynucleotide of any of claims 20 to 22.

24. A vector according to claim 23, which is an expression vector.

25. A cell comprising a vector according to claims 23 or 24.

26. A method for the preparation of an EMR2 polypeptide-type polypeptide which is
EMR2 polypeptide protein comprising the amino acid sequence of SEQ ID NO. 2 or SEQ ID NO. 20;
a polypeptide which has substantial identity therewith and which retains an EMR2 polypeptide biological activity; or
a fragment of such an EMR2 polypeptide protein or substantially identical polypeptide, and which fragment retains an EMR2 polypeptide biological activity;
or a substance as defined in any of claims 15 to 19 comprising culturing a cell according to claim 25 which contains an expression vector according to claim 24, and collecting the expressed polypeptide.

27. A method according to claim 26, wherein the vector is so designed as to express the polypeptide in secretory form.

28. A preparation of an EMR2 polypeptide-type polypeptide which is
EMR2 polypeptide protein comprising the amino acid sequence of SEQ ID NO. 2 or SEQ ID NO. 20;
a polypeptide which has substantial identity therewith and which retains an EMR2 polypeptide biological activity; or
a fragment of such an EMR2 polypeptide protein or substantially identical polypeptide, and which fragment retains an EMR2 polypeptide biological activity;
or a substance as defined in any of claims 15 to 19, suitable to modify the activity of EMR2 in vivo.

29. A preparation according to claim 28 which is a pharmaceutical preparation.

30. A preparation according to claim 27 or 28 for blocking the effect of EMR2 in vivo.

31. A preparation according to any of claims 28 to 30 for the treatment of a condition selected from: acute inflammation caused by injury or infection, such as meningitis and pneumonia; chronic inflammation, such as rheumatoid arthritis, inflammatory bowel disease (Crohn's disease, ulcerative colitis), chronic tissue damages; septic shock; repair and auto-immune disease processes; atherosclerosis; diabetes; Alzheimer's disease; and processes such as killing of targets by degranulation; chemotaxis and leukocyte recruitment; induction and effector mechanism of innate and acquired autoimmunity.

32. A preparation according to any of claims 28 to 30 for the treatment of a condition involving: clotting fibrinolysis; intravascular coagulation; thrombosis and embolism; wound repair and angiogenesis.

33. A preparation according to any of claims 28 to 30 for the treatment of a condition involving: haematopoiesis and blood disorders such as neutropenia and agranulocytosis as well as myeloid leukaemia; and anaemia.

34. A preparation according to any of claims 28 to 30 for the treatment of a condition involving: the migration, retention and activation/de-activation of phagocytes.

35. A preparation according to any of claims 28 to 30 for the treatment of a condition selected from: general disorders of connective tissue such as wound healing, vascular malfunction and congenital diseases such as hereditary hemorrhagic telangiectasia (HHT) and Marfan syndrome.

36. A preparation according to any of claims 28 to 30 for the control of tumour formation and metastasis.

37. A preparation according to any of claims 28 to 30 for the treatment of Mφ giant cells in bacterially-induced granuloma.

38. A substrate, such as beads, carrying an EMR2 polypeptide-type polypeptide which is
EMR2 polypeptide protein comprising the amino acid sequence of SEQ ID NO. 2 or SEQ ID NO. 20;
a polypeptide which has substantial identity therewith and which retains an EMR2 polypeptide biological activity; or
a fragment of such an EMR2 polypeptide protein or substantially identical polypeptide, and which fragment retains an EMR2 polypeptide biological activity;
or a substance as defined in any of claims 15 to 19;
said polypeptide optionally being attached to said substrate by means of a linker sequence.

39. A method for the diagnosis of EMR2 expression comprising detecting EMR2 in a sample with an antibody specific for EMR2 and assaying the extent of binding of said antibody.

40. A method for the diagnosis of EMR2 expression comprising assaying identity between EMR2 DNA in a sample and a polynucleotide according to any of claims 20 to 22, or a fragment thereof.

41. A pharmaceutical composition comprising
an EMR2 polypeptide-type polypeptide which is
EMR2 polypeptide protein comprising the amino acid sequence of SEQ ID NO. 2 or 20;
a polypeptide which has substantial identity therewith and which retains an EMR2 polypeptide biological activity; or
a fragment of such an EMR2 polypeptide protein or substantially identical polypeptide, and which fragment retains an EMR2 polypeptide biological activity;
in the preparation of a diagnostic agent for use in a method of diagnosis of a condition or disease associated with EMR2 polypeptide.


SEQUENCE LISTING (22 sequences)

SEQ ID NO: 1 - DNA, 6068 bp, Homo sapiens
  CDS (70)..(2541)
  sig_peptide (70)..(138)
  5′UTR (1)..(69)
  3′UTR (2542)..(6068)
cggagacgggacagccctgtcccactcactctttcccctgctgctcctgccggcagctcagctggaaccatgggaggccgcgtctttctcgtctttctcgcattctgtgtctggctgactctgccgggagctgaaacccaggactccaggggctgtgcccggtggtgccctcaggactcctcgtgtgtcaatgccaccgcctgtcgctgcaatccagggttcagctctttttctgagatcatcaccacccccatggagacttgtgacgacatcaacgagtgtgcaacactgtcgaaagtgtcatgcggaaaattctcggactgctggaacacagaggggagctacgactgcgtgtgcagcccaggatatgagcctgtttctggggcaaaaacattcaagaatgagagcgagaacacgtgtcaagatgtggacgaatgtcagcagaacccaaggctctgtaaaagctacggcacctgcgtcaacaccctcggcagctacacgtgccagtgcctgcctggcttcaagctcaaacctgaggacccgaagctctgcacagatgtgaatgaatgcacctccggacaaaacccatgccacagctccacccactgcctcaacaacgtgggcagctatcagtgccgctgccgcccgggctggcaaccgattccggggtcccccaatggcccaaacaataccgtctgtgaagatgtggacgagtgcagctccgggcagcatcagtgtgacagctccaccgtctgcttcaacaccgtgggttcatacagctgccgctgccgcccaggctggaagcccagacacggaatcccgaataaccaaaaggacactgtctgtgaagatatgactttctccacctggaccccgccccctggagtccacagccagacgctttcccgattcttcgacaaagtccaggacctgggcagagactacaagccaggcttggccaataacaccatccagagcatcttacaggcgctggatgagctgctggaggcccctggggacctggagaccctgccccgcttacagcagcactgtgtggccagtcacctgctggatggcctagaggatgtcctcagaggcctgagcaagaacctttccaatgggctgttgaacttcagttatcctgcaggcacagaattgtccctggaggtgcagaagcaagtagacaggagtgtcaccttgagacagaatcaggcagtgatgcagctcgactggaatcaggcacagaaatctggtgacccaggcccttctgtggtgggccttgtctccattccagggatgggcaagttgctggctgaggcccctctggtcctggaacctgagaagcagatgcttctgcatgagacacaccagggcttgctgcaggacggctcccccatcctgctctcagatgtgatctctgcctttctgagcaacaacgacacccaaaacctcagctccccagttaccttcaccttctcccaccgttcagtgatcccgagacagaaggtgctctgtgtcttctgggagcatggccagaatggatgtggtcactgggccaccacaggctgcagcacaataggcaccagagacaccagcaccatctgccgttgcacccacctgagcagctttgccgtcctcatggcccactacgatgtgcaggaggaggatcccgtgctgactgtcatcacctacatggggctgagcgtctctctgctgtgcctcctcctggcggccctcacttttctcctgtgtaaagccatccagaacaccagcacctcactgcatctgcagctctcgctctgcctcttcctggcccacctcctcttcctcgtggcaattgatcaaaccggacacaaggtgctgtgctccatcatcgccggtaccttgcactatctctacctggccaccttcacctggatgctgctggaggccctgtacctcttcctcactgcacggaacctgacggtggtcaactactcaagcatcaacagattcatgaagaagctcatgttccctgtgggctacggagtcccagctgtgacagtggccatttctgcagcctccaggcctcacctttatggaacaccttcccgctgctggctccaaccagaaaagggatttatatggggcttccttggacctgtctgcgccatcttctctgtgaatttagttctctttctggtgactctctggattttgaaaaacagactctcctccctcaatagtgaagtgtccaccctccggaacacaaggatgctggcatttaaagcgacagctcagctgttcatcctgggctgcacgtggtgtctgggcatcttgcaggtgggtccggctgcccgggtcatggcctacctcttcaccatcatcaacagcctgcagggtgtcttcatcttcctggtgtactgcctcctcagccagcaggtccgggagcaatatgggaaatggtccaaagggatcaggaaattgaaaactgagtctgagatgcacacactctccagcagtgctaaggctgacacctccaaacccagcacggttaactagaaaaatcttctgaataagatcttccctctttgccggtggaaaatctgaacaatctttgagccatctagaggggaaagaaaagactttgttctgtgtgtttcaagaaattcaccatgtcagcaatatgaaggatgttatggaaggcgtgcttggcattcaattcctgcagaaaccggaaatcttccatgccctgcaatgtgctcatcaaactctcagcatatggacggccagctgtggcccatatcttggtcactctgaagcacaatatttatgaagctatagaacgttaagacctctttcacagcctctccttcctacaaagactcctccaaatcttaaaatgaagcaggaaaacaagcctaagaggactttcataccgacaacatctgaaaggactagaatgttcacaccacgatctggatttcttaattttttgtttttgtttttgttgttctctagttctacgggtttgattatttagtcatgtgaaaaatattgattactcacacatagatcaagagagacacggctcctgccttcatggagcttttaggggaaaatgaagtggctcttgcagctagagttgactcagaagccgaaattcctagaaatcaggtttctactgctaggcaattgaagtataaactattttataaacactgtcttctttcatcttcacaccaacatgcagaaaagtttctaatctcagatcagggatgtgcaacaaattccatttcaaaggaatgacctgcaaaactcctaaatattccaagcaaatgcccttaaccctgtctgttatctgctttccttgaacagaaattctacatgaccataaaacctcgaagatgggtatggcacagttcatgccctgtaatcctagcactttgggagggtgaggcaggaggatggctcaagcccaggagtttgagaccagtgtgggcaacagagtgagaaccatctctaccccaaaaaaaaattaaaaattagccaagcatggtgatgatataggagttaaggagaaatcatttaggcaaatagcaagggtaggaagtcctcagtgaggttttctgtttaatgaaaagcagcccccaaaatcattttcttttctaacaaagaacagcctgtaaaatcgagctgcagacatagacaagcaagctggaagcttccacgggtgaatgctggcaactgtgccaataggaaaaagctacctagactaggcatgtccaaaatggcggctccaagttcccttctctttgccagccatgtgtacagtaaaaagcaggcaacatagtgtcagccaaagctcatttgcataataagattagggtggggtggccagctcacataggggtaggccctaggtaaatcagacaccgccttctcaagcctgtctataaaatctggtacactatgacgagggtcagatttcccattcagacgcccctctcccatgcaagagaaagagctgttctcctttctctttcttttgcctattaaacctctgctcctggccaggcacagtggctcacgcctataatcccagcactttgggaggctgaggtggtcagatcacctaaggtcaggagttcaagaccagcctggtcaacatggtgaaatcttgtctctagtaaaaatacaaaaatatatgaaatctcacatagatgataatactaagttccaaaagcaactcaacctggtagattctaattttttttgaggcagggtcttgctttgtcacccatgctggagtacaatggcacaaacactgctcactgcagcctcgacctcccaaggcctaagcaatcctcctgcctcagtccccctccaagtagctgaaactacaggtgtgtaccaccacacctggctaatttttgtattttttgtagagacgtgggtctcactatgctgcccaggctcaggtcttaatctcctgagctcaggcaatccgcaggcctcagcctccctaagtgcggggattacaggcttgagccactgcacctagcctctatttgttttacaaaagagaaattgagatcctgaatgttaagtgacttgcctgaggccatcccactaacaggagccagggttaggattcaaaccccatccaactggtcccagagctggagcttcttgcactgccctacactacctaccatctccatcctctgggcacctttttataagaaccaaaacattacagagcattgctttgtcaactcagctgggaacatttcccagtgcaactcacatttttcactgctctgtgcctgtctgtataagctcaatgagtattgatttaggggctttggagaactttgaatgctaccccccaagtaaccattattggcaacctggtacctctacttttagccatttctccttctctataaacagtgcagaagtaacccacttggtaacaggcatccttgccaagcctccaccactaggtcagtgtaagaattaaagaaagaggaaagaaacacgaaaagtggcttgatggttaagacaggtttattttagagaaaacacacctgagaggggctgctggctgaattaggttagagtcttttctacagactaagagtgtttaaggatttagggtgggagagtttcttagaggcttggactgcttctgtgtttttttgttgtgcttatatgggagggagagtggtgtgtttgcttttatacatttttctgcagctgtaggcataccccccaagtctgcttttagcttccctattttagtgcacctggagggaaaggaatgtgcttattaaggcccactgttttactggggcccattgtatgagggtgaagtttggcagttacccaagagacttttcctccaccttcctctgtgcccgagctgttttatctgcattttactgtctgctttttttggctgcttatagtttttaaaaaagtaatttccttaaatccagaaggctaaaaatgaagctgaaacttaaagtggcggtgtttgtccaaaataacggggctcctgctctgccagtcagtaccctcaagtcactcctgatcctcaacctccatgcctaagactggttcaagagaccacataatatctgccttttattacatacatgatgggtgcatgggattctgcatgccctttgcttgatatagactgctaaggtgagatggggaatatcagagtcagctgctgcttgaggaagcagaacacacagctggaggcttggaacatgtgggtccctatgagtgtagagcccatatccccatagagtctacctagagcaggggtcgccaaatgttttcttaaagagcctgatagtgtatatgttaggctttgtgagccaggtatttacagcaactcaattctaccactgtggtatgaaaacagctatagacaatcataaatgaatgatcatggctatgttttaataaaactttacagacactgaacttgaacttccattgtgatatgaaaacagctatagacaatcataaatgaatgatcatggctatgttttaataaaactttatggaca

SEQ ID NO: 2 - protein, 823 aa, Homo sapiens [sequence omitted]

SEQ ID NO: 3 - DNA, 27 bp, Homo sapiens
ccatcctaatacgactcactatagggc

SEQ ID NO: 4 - DNA, 23 bp, Homo sapiens
actcactatagggctcgagcggc

SEQ ID NO: 5 - DNA, 29 bp, Homo sapiens
ctggtgttctggatggctttacacaggag

SEQ ID NO: 6 - DNA, 22 bp, Homo sapiens
tgcacatcgtagtgggccatga

SEQ ID NO: 7 - DNA, 22 bp, Homo sapiens
aggtgctctgtgtcttctggga

SEQ ID NO: 8 - DNA, 22 bp, Homo sapiens
gtgctgtgctccatcatcgccg

SEQ ID NO: 9 - DNA, 26 bp, Homo sapiens
ttccaccggcaaagagggaagatctt

SEQ ID NO: 10 - DNA, 27 bp, Homo sapiens
gacagtggccatttctgcagcctccag

SEQ ID NO: 11 - DNA, 22 bp, Homo sapiens
aggtgctctgtgtcttctggga

SEQ ID NO: 12 - DNA, 25 bp, Homo sapiens
ttctggttggagccagcgggaaggt

SEQ ID NO: 13 - DNA, 26 bp, Homo sapiens
cgcaccacaagaaagtagagctccag

SEQ ID NO: 14 - DNA, 25 bp, Homo sapiens
gactggaagctgaccctgatcacca

SEQ ID NO: 15 - DNA, 34 bp, Homo sapiens
acgggatcctcctcctgcacatcgtgggccatgt

SEQ ID NO: 16 - DNA, 50 bp, Homo sapiens
aaccatgggaggccgcgtctttctcgtctttctcgcattctgtgtctggc

SEQ ID NO: 17 - DNA, 22 bp, Homo sapiens
aggtgctctgtgtcttctggga

SEQ ID NO: 18 - DNA, 30 bp, Homo sapiens
ttccaccggcaaagagggaagatcttattc

SEQ ID NO: 19 - DNA, 3517 bp, Homo sapiens
  CDS (70)..(2541)
  3′UTR (2542)..(3517)
  5′UTR (1)..(69)
  sig_peptide (70)..(138)
cggagacgggacagccctgtcccactcactctttcccctgctgctcctgccggcagctcagctggaaccatgggaggccgcgtctttctcgtctttctcgcattctgtgtctggctgactctgccgggagctgaaacccaggactccaggggctgtgcccggtggtgccctcaggactcctcgtgtgtcaatgccaccgcctgtcgctgcaatccagggttcagctctttttctgagatcatcaccacccccatggagacttgtgacgacatcaacgagtgtgcaacactgtcgaaagtgtcatgcggaaaattctcggactgctggaacacagaggggagctacgactgcgtgtgcagcccaggatatgagcctgtttctggggcaaaaacattcaagaatgagagcgagaacacgtgtcaagatgtggacgaatgtcagcagaacccaaggctctgtaaaagctacggcacctgcgtcaacaccctcggcagctacacgtgccagtgcctgcctggcttcaagctcaaacctgaggacccgaagctctgcacagatgtgaatgaatgcacctccggacaaaacccatgccacagctccacccactgcctcaacaacgtgggcagctatcagtgccgctgccgcccgggctggcaaccgattccggggtcccccaatggcccaaacaataccgtctgtgaagatgtggacgagtgcagctccgggcagcatcagtgtgacagctccaccgtctgcttcaacaccgtgggttcatacagctgccgctgccgcccaggctggaagcccagacacggaatcccgaataaccaaaaggacactgtctgtgaagatatgactttctccacctggaccccgccccctggagtccacagccagacgctttcccgattcttcgacaaagtccaggacctgggcagagactacaagccaggcttggccaataacaccatccagagcatcttacaggcgctggatgagctgctggaggcccctggggacctggagaccctgccccgcttacagcagcactgtgtggccagtcacctgctggatggcctagaggatgtcctcagaggcctgagcaagaacctttccaatgggctgttgaacttcagttatcctgcaggcacagaattgtccctggaggtgcagaagcaagtagacaggagtgtcaccttgagacagaatcaggcagtgatgcagctcgactggaatcaggcacagaaatctggtgacccaggcccttctgtggtgggccttgtctccattccagggatgggcaagttgctggctgaggcccctctggtcctggaacctgagaagcagatgcttctgcatgagacacaccagggcttgctgcaggacggctcccccatcctgctctcagatgtgatctctgcctttctgagcaacaacgacacccaaaacctcagctccccagttaccttcaccttctcccaccgttcagtgatcccgagacagaaggtgctctgtgtcttctgggagcatggccagaatggatgtggtcactgggccaccacaggctgcagcacaataggcaccagagacaccagcaccatctgccgttgcacccacctgagcagctttgccgtcctcatggcccactacgatgtgcaggaggaggatcccgtgctgactgtcatcacctacatggggctgagcgtctctctgctgtgcctcctcctggcggccctcacttttctcctgtgtaaagccatccagaacaccagcacctcactgcatctgcagctctcgctctgcctcttcctggcccacctcctcttcctcgtggcaattgatcaaaccggacacaaggtgctgtgctccatcatcgccggtaccttgcactatctctacctggccaccttcacctggatgctgctggaggccctgtacctcttcctcactgcacggaacctgacggtggtcaactactcaagcatcaacagattcatgaagaagctcatgttccctgtgggctacggagtcccagctgtgacagtggccatttctgcagcctccaggcctcacctttatggaacaccttcccgctgctggctccaaccagaaaagggatttatatggggcttccttggacctgtctgcgccatcttctctgtgaatttagttctctttctggtgactctctggattttgaaaaacagactctcctccctcaatagtgaagtgtccaccctccggaacacaaggatgctggcatttaaagcgacagctcagctgttcatcctgggctgcacgtggtgtctgggcatcttgcaggtgggtccggctgcccgggtcatggcctacctcttcaccatcatcaacagcctgcagggtgtcttcatcttcctggtgtactgcctcctcagccagcaggtccgggagcaatatgggaaatggtccaaagggatcaggaaattgaaaactgagtctgagatgcacacactctccagcagtgctaaggctgacacctccaaacccagcacggttaactagaaaaatcttctgaataagatcttccctctttgccggtggaaaatctgaacaatctttgagccatctagaggggaaagaaaagactttgttctgtgtgtttcaagaaattcaccatgtcagcaatatgaaggatgttatggaaggcgtgcttggcattcaattcctgcagaaaccggaaatcttccatgccctgcaatgtgctcatcaaactctcagcatatggacggccagctgtggcccatatcttggtcactctgaagcacaatatttatgaagctatagaacgttaagacctctttcacagcctctccttcctacaaagactcctccaaatcttaaaatgaagcaggaaaacaagcctaagaggactttcataccgacaacatctgaaaggactagaatgttcacaccacgatctggatttcttaattttttgtttttgtttttgttgttctctagttctacgggtttgattatttagtcatgtgaaaaatattgattactcacacatagatcaagagagacacggctcctgccttcatggagcttttaggggaaaatgaagtggctcttgcagctagagttgactcagaagccgaaattcctagaaatcaggtttctactgctaggcaattgaagtataaactattttataaacactgtcttctttcatcttcacaccaacatgcagaaaagtttctaatctcagatcagggatgtgcaacaaattccatttcaaaggaatgacctgcaaaactcctaaatattccaagcaaatgcccttaaccctgtctgttatctgctttccttgaacagaaattctacatgaccataaaacctcgaagatgggtatggcacagttcatgccctgtaatcctagcactttgggagggtgaggcaggaggatggctcaagcccaggagtttgagaccagtgtgggcaacagagtgagaaccatctctaccccaaaaaaaaa

SEQ ID NO: 20 - protein, 800 aa, Homo sapiens
QDSRGCARWCPQDSSCVNATACRCNPGFSSFSEIITTPMETCDDINECATLSKVSCGKFSDCWNTEGSYDCVCSPGYEPVSGAKTFKNESENTCQDVDECQQNPRLCKSYGTCVNTLGSYTCQCLPGFKLKPEDPKLCTDVNECTSGQNPCHSSTHCLNNVGSYQCRCRPGWQPIPGSPNGPNNTVCEDVDECSSGQHQCDSSTVCFNTVGSYSCRCRPGWKPRHGIPNNQKDTVCEDMTFSTWTPPPGVHSQTLSRFFDKVQDLGRDYKPGLANNTIQSILQALDELLEAPGDLETLPRLQQHCVASHLLDGLEDVLRGLSKNLSNGLLNFSYPAGTELSLEVQKQVDRSVTLRQNQAVMQLDWNQAQKSGDPGPSVVGLVSIPGMGKLLAEAPLVLEPEKQMLLHETHQGLLQDGSPILLSDVISAFLSNNDTQNLSSPVTFTFSHRSVIPRQKVLCVFWEHGQNGCGHWATTGCSTIGTRDTSTICRCTHLSSFAVLMAHYDVQEEDPVLTVITYMGLSVSLLCLLLAALTFLLCKAIQNTSTSLHLQLSLCLFLAHLLFLVAIDQTGHKVLCSIIAGTLHYLYLATFTWMLLEALYLFLTARNLTVVNYSSINRFMKKLMFPVGYGVPAVTVAISAASRPHLYGTPSRCWLQPEKGFIWGFLGPVCAIFSVNLVLFLVTLWILKNRLSSLNSEVSTLRNTRMLAFKATAQLFILGCTWCLGILQVGPAARVMAYLFTIINSLQGVFIFLVYCLLSQQVREQYGKWSKGIRKLKTESEMHTLSSSAKADTSKPSTVN

SEQ ID NO: 21 - protein, 835 aa, Homo sapiens [sequence omitted]

SEQ ID NO: 22 - protein, 850 aa, Homo sapiens
MRGFNLLLFWGCCVMHSWEGHIRPTRKPNTKGNNCRDSTLCPAYATCTNTVDSYYCTCKQGFLSSNGQNHFKDPGVRCKDIDECSQSPPPCGPNSSCKNTEGSYDCVCSPGYEPVSGTKTFKNESENTCQDVDECQQNPRLCKSYGTCVNTLGSYTCQCLPGFKFIPEDPKVCTDVNECTSGQNPCHSSTHCLNNVGSYQCRCRPGWQPIPGSPNGPNNTVCEDVDECSSGQHQCDSSTVCFNTVGSYSCRCRPGWKPRHGIPNNQKDTVCEDMTFSTWTPPPGVHSQTLSRFFDKVQDLGRDSKTSSAEVTIQNVIKLVDELMEAPGDVEALAPPVRHLIATQLLSNLEDIMRILAKSLPKGPFTYISPSNTELTLMIQERGDKNVTMGQSSARMKLNWAVAAGAEDPGPAVAGILSIQNMTTLLANASLNLHSKKQAELEEIYESSIRGVQLRRLSAVNSIFLSHNNTKELNSPILFAFSHLESSDGEAGRDPPAKDVMPGPRQELLCAFWKSDSDRGGHWATEGCQVLGSKNGSTTCQCSHLSSFAILMAHYDVEDWKLTLITRVGLALSLFCLLLCILTFLLVRPIQGSRTTIHLHLCICLFVGSTIFLAGIENEGGQVGLRCRLVAGLLHYCFLAAFCWMSLEGLELYFLVVRVFQGQGLSTRWLCLIGYGVPLLIVGVSAAIYSKGYGRPRYCWLDFEQGFLWSFLGPVTFIILCNAVIFVTTVWKLTQKFSEINPDMKKLKKARALTITAIAQLFLLGCTWVFGLFIFDDRSLVLTYVFTILNCLQGAFLYLLHCLLNKKVREEYRKWACLVAGGSKYSEFTSTTSGTGHNQTRALRASESGI